SEQUENCE LISTING (12 sequences)

SEQ ID NO: 1 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 11 aa, synthetic construct
RASQGIRNYLA

SEQ ID NO: 3 - protein, 7 aa, synthetic construct
AASTLQS

SEQ ID NO: 4 - protein, 9 aa, synthetic construct
QRYNRAPYT

SEQ ID NO: 5 - protein, 5 aa, synthetic construct
DYAMH

SEQ ID NO: 6 - protein, 17 aa, synthetic construct
AITWNSGHIDYADSVEG

SEQ ID NO: 7 - protein, 12 aa, synthetic construct
VSYLSTASSLDY

SEQ ID NO: 8 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 9 - protein, 131 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTAS

SEQ ID NO: 10 - protein, 450 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 11 - protein, 107 aa, synthetic construct [sequence omitted]

SEQ ID NO: 12 - protein, 121 aa, synthetic construct [sequence omitted]

(19) United States
(12) Patent Application Publication
(10) Pub. No.: US 2025/0281608 A1
(43) Pub. Date: Sep. 11, 2025

(54) STABLE FORMULATIONS FOR TNF-alpha ANTIBODIES AND USES THEREOF

(71) Applicant: Zenas BioPharma, Inc., Waltham, MA (US)

(72) Inventors: Gan Wei, Waltham, MA (US); Boxu Yan, Waltham, MA (US); Darren Mushrush, Waltham, MA (US); Xiaodong Wang, Waltham, MA (US); Rachel Kirk, Waltham, MA (US); Stephen Wax, Waltham, MA (US)

(21) Appl. No.: 19/071,375

(22) Filed: Mar. 5, 2025

Related U.S. Application Data
(60) Provisional application No. 63/561,521, filed on Mar. 5, 2024.
(60) Provisional application No. 63/658,731, filed on Jun. 11, 2024.
(60) Provisional application No. 63/720,874, filed on Nov. 15, 2024.

(51) Int. Cl. A61K 39/395; A61K 39/00; A61K 47/12; A61K 47/20; A61K 47/22; A61K 47/26; C07K 16/24

(52) U.S. Cl. CPC A61K 39/39591; A61K 47/12; A61K 47/20; A61K 47/22; A61K 47/26; C07K 16/241; A61K 2039/505; A61K 2039/54; A61K 2039/545; C07K 2317/33; C07K 2317/52; C07K 2317/71; C07K 2317/92

(57) ABSTRACT

Antibodies, fragments thereof and fusion proteins that specifically bind to TNFα, are described, as well as stable formulations comprising such antibodies. Such antibodies, fusion proteins and fragments thereof are useful for the treatment and diagnosis of various autoimmune diseases.

### CROSS REFERENCE TO RELATED APPLICATIONS

This application claims priority to U.S. Provisional Application No. 63/561,521, filed Mar. 5, 2024, U.S. Provisional Application No. 63/658,731, filed on Jun. 11, 2024, and U.S. Provisional Application No. 63/720,874, filed on Nov. 15, 2024.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Mar. 2, 2025, is named “ZEN-015US1_SL” and is 12,810 bytes in size.

### BACKGROUND

TNFα is a potent inflammatory cytokine, produced primarily by activated monocytes, macrophages, and T cells, as a cell surface protein. After being activated, TNFα is released and binds to receptors on TNFα-responsive cells to enhance the inflammatory and immune response to environmental stimuli such as foreign antigens. Elevated expression of TNFα has been linked to a number of inflammatory diseases, and inhibition of TNFα signaling has been validated as a therapeutic approach to treat several diseases.

TNFα is synthesized as a transmembrane protein on the plasma membrane, and subsequently may be proteolytically processed by TNFα-converting enzyme (TACE), liberating soluble TNFα homotrimer protein (Sedger and McDermott, Cytokine & Growth Factor Reviews (2014) 25:453-72). TNFα is a potent mediator of inflammation and is implicated in the pathogenesis of inflammatory and autoimmune diseases (Kalliolias and Ivashkiv, Nat Rev Rheumatol. (2016) 12:49-62).

The FDA and other health agencies have approved five anti-TNFα therapies to treat diseases, including rheumatoid arthritis, psoriatic arthritis, ankylosing spondylitis, Crohn's disease, psoriasis, ulcerative colitis, hidradenitis suppurativa, and uveitis. Among those approved therapies are Remicade® (infliximab), Simponi® (golimumab) and Humira® (adalimumab), monoclonal antibodies that target TNFα for inhibition.

Because mAbs depend on their Fc region for eliciting certain immune reactions, a way to improve their action is to engineer this portion of the antibody. For instance, for most therapeutic applications, a long serum half-life is desirable as it would decrease the need for repetitive injections of the molecule to achieve a therapeutically relevant serum concentration.

Fc domains can be engineered to increase binding affinity to the receptor FcRn. FcRn is present inside lysosomes in endothelial cells lining the blood vessels and functions to rescue antibodies from the degradation that makes most proteins short-lived in circulation. As a result of interactions with FcRn antibodies can have prolonged half-lives allowing less frequent dosing for antibody drugs than most other biologics.

A recombinant human mAb directed at human TNFα. The TNFα-binding region within the fragment variable (Fv) domain has an identical amino acid sequence to adalimumab (Humira®). The fragment crystallizable (Fc) domain consists of a hybrid immunoglobulin (IgG1/2) constant region containing amino acid substitutions for extended half-life.

### SUMMARY OF INVENTION

The present invention provides, among other things, a stable formulation for anti-TNFα antibodies and methods of treating autoimmune diseases using the same that require less frequent dosing. As described herein, the present disclosure is based, in part, on the identification of a stable formulation comprising an anti-TNFα antibody at a concentration of greater than 50 mg/mL, wherein less than 5% the anti-TNFα antibody exists as high molecular weight (HMW) species. This is significant because a stable formulation with a high concentration of the anti-TNFα antibody allows administration of a smaller volume to patients. Additionally, the stable formulation of the present invention can be stored for longer period of time without sacrificing the stability of the anti-TNFα antibody. The improved in vivo half-life (t_{1/2}) of the anti-TNFα antibodies disclosed herein furthermore enables the efficacious treatment of autoimmune diseases using a less frequent dosing interval as compared to existing treatment regimens.

In one aspect, the present invention provides a stable formulation, comprising an anti-TNFα antibody at a concentration of at least 50 mg/mL, wherein the anti-TNFα antibody comprises a light chain variable region comprising a LCDR1 of SEQ ID NO: 2, a LCDR2 of SEQ ID NO: 3, and a LCDR3 of SEQ ID NO: 4; a heavy chain variable region comprising a HCDR1 of SEQ ID NO: 5, a HCDR2 of SEQ ID NO: 6, and a HCDR3 of SEQ ID NO: 7, and an Fc region comprising M428L and N434S substitutions. In some embodiments, the light chain variable region comprises SEQ ID NO: 11 and the heavy chain variable region comprises SEQ ID NO: 12. In some embodiments, the anti-TNFα antibody comprises a light chain of SEQ ID NO: 1 and a heavy chain of SEQ ID NO: 2.

In some embodiments, the concentration of the anti-TNFα antibody is between 50 mg/mL and 200 mg/mL. In some embodiments, the concentration of the anti-TNFα antibody is between 90 mg/mL and 110 mg/mL. In some embodiments, the concentration of the anti-TNFα antibody is 100 mg/mL. In some embodiments, the concentration of anti-TNFα antibody is between 135 mg/mL and 165 mg/mL. In some embodiments, the concentration of the anti-TNFα antibody is 150 mg/mL. In some embodiments, the concentration of the anti-TNFα antibody is at least 50 mg/mL, at least 100 mg/mL, at least 150 mg/mL or at least 200 mg/mL.

In some embodiments, the formulation further comprises a buffer. In some embodiments, the buffer comprises a histidine buffer. In some embodiments, histidine buffer is present at a concentration of less than 60 mM. In some embodiments, histidine buffer is present at a concentration of 5 mM to 40 mM. In some embodiments, histidine buffer is present at a concentration of 20 mM. In some embodiments, the buffer comprises an acetate buffer. In some embodiments, acetate is present at a concentration of less than 60 mM. In some embodiments, acetate is present at a concentration of 5 mM to 40 mM. In some embodiments, acetate is present at a concentration of 20 mM.

In some embodiments, the formulation comprises a pH of 5.0 to 6.0. In some embodiments, the pH 5.5.

In some embodiments, the formulation further comprises a sugar. In some embodiments, the sugar is present at a concentration of 2% (w/v) to 10% (w/v). In some embodiments, the sugar comprises sucrose or trehalose. In some embodiments, the sugar comprises sucrose. In some embodiments, sucrose is present at a concentration of 4% (w/v). In some embodiments, sucrose is present at a concentration of 7% (w/v).

In some embodiments, the formulation further comprises a polyol. In some embodiments, the polyol is present at a concentration of 2% (w/v) to 10% (w/v). In some embodiments, the polyol comprises sorbitol or mannitol. In some embodiments, the polyol comprises sorbitol. In some embodiments, sorbitol is present at a concentration of 4.5% (w/v).

In some embodiments, the formulation further comprises an antioxidant. In some embodiments, the antioxidant comprises L-methionine. In some embodiments, L-methionine is present at a concentration of 5 mM to 20 mM. In some embodiments, L-methionine is present at a concentration of 10 mM.

In some embodiments, the formulation further comprises a surfactant.

In some embodiments, the surfactant comprises polysorbate 80 or poloxamer-188. In some embodiments, the surfactant comprises polysorbate 80. In some embodiments, polysorbate 80 is present at a concentration of 0.001% (w/v) to 0.1% (w/v). In some embodiments, polysorbate 80 is 0.01% (w/v) to 0.06% (w/v). In some embodiments, the concentration of polysorbate 80 is 0.02% (w/v).

In some embodiments, the formulation is substantially free of salt.

In some embodiments, the formulation further comprises an amino acid. In some embodiments, the amino acid is present at a concentration of 5 mM to 150 mM. In some embodiments, the amino acid comprises arginine or proline.

In one aspect, the present invention provides a stable formulation comprising (i) 100 mg/mL of the an anti-TNFα antibody, (ii) 20 mM of histidine buffer, (iii) 4.5% (w/v) of sorbitol, (iv) 10 mM of L-methionine, (v) 0.02% (w/v) of polysorbate 80, wherein the anti-TNFα antibody comprises a light chain variable region comprising a LCDR1 of SEQ ID NO: 2, a LCDR2 of SEQ ID NO: 3, and a LCDR3 of SEQ ID NO: 4; a heavy chain variable region comprising a HCDR1 of SEQ ID NO: 5, a HCDR2 of SEQ ID NO: 6, and a HCDR3 of SEQ ID NO: 7, and an Fc region comprising M428L and N434S substitutions, and wherein the pH is 5.5. In some embodiments, formulation comprises an osmolality of between 310 mOsmol/kg and 410 mOsmol/kg. In some embodiments, the formulation is substantially free of salt.

In one aspect, the present invention provides a stable formulation comprising (i) 150 mg/mL of an anti-TNFα antibody, (ii) 20 mM of histidine buffer, (iii) 4.5% (w/v) of sorbitol, (iv) 10 mM of L-methionine, (v) 0.02% (w/v) of polysorbate 80, wherein the anti-TNFα antibody comprises a light chain variable region comprising a LCDR1 of SEQ ID NO: 2, a LCDR2 of SEQ ID NO: 3, and a LCDR3 of SEQ ID NO: 4; a heavy chain variable region comprising a HCDR1 of SEQ ID NO: 5, a HCDR2 of SEQ ID NO: 6, and a HCDR3 of SEQ ID NO: 7, and an Fc region comprising M428L and N434S substitutions, and wherein the pH is 5.5. In some embodiments, the formulation comprises an osmolality of 275 mOsmol/kg to 375 mOsmol/kg. In some embodiments, the formulation is substantially free of salt.

In some embodiments, the percentage of monomer is equal to or greater than 90%, equal to or greater than 91%, equal to or greater than 92%, equal to or greater than 93%, equal to or greater than 94%, equal to or greater than 95%, equal to or greater than 96%, equal to or greater than 97%, equal to or greater than 98%, equal to or greater than 99%, equal to or greater than 99.5%. In some embodiments, the percentage of high molecular weight species is less than or equal to 5%, less than or equal to 4%, less than or equal to 3%, less than or equal to 2%, less than or equal to 1% or less than or equal to 0.5%.

In some embodiments, storage of the formulation at 2-8° C. for at least 3 months results in the formulation comprising less than 5% aggregates, and greater than 90% intact antibody. In some embodiments, storage of the formulation at 25° C. for at least 3 months results in the formulation comprising less than 5% aggregates, and greater than 90% intact antibody. In some embodiments, storage of the formulation at 40° C. for at least 3 months results in the formulation comprising less than 5% aggregates, and greater than 90% intact antibody.

In one aspect, the present invention comprises a method of treating an autoimmune disease in a subject comprising administering to a subject in need of treatment the stable formulation of any one of the preceding embodiments.

In one aspect, the present invention provides a method of treating an autoimmune disease in a subject comprising, administering to the subject an anti-TNFα antibody at a dose of at least 20 mg no more frequently than once every other week, wherein the anti-TNFα antibody comprises a light chain variable region comprising a LCDR1 of SEQ ID NO: 2, a LCDR2 of SEQ ID NO: 3, and a LCDR3 of SEQ ID NO: 4, a heavy chain variable region comprising a HCDR1 of SEQ ID NO: 5, a HCDR2 of SEQ ID NO: 6, and a HCDR3 of SEQ ID NO: 7, and an Fc region comprising M428L and N434S substitutions. In some embodiments, the light chain variable region comprises SEQ ID NO: 11 and the heavy chain variable region comprises SEQ ID NO: 12. In some embodiments, the anti-TNFα antibody comprises a light chain of SEQ ID NO: 1 and a heavy chain of SEQ ID NO: 2.

In some embodiments, the anti-TNFα antibody is administered no more frequently than once every three weeks, no more frequently than once every four weeks, no more frequently than once every month, no more frequently than once every five weeks, no more frequently than once every six weeks, no more frequently than once every seven weeks, no more frequently than once every eight weeks or no more frequently than once every other month.

In some embodiments, the anti-TNFα antibody is administered at a dose of 40 mg to 160 mg.

In some embodiments, anti-TNFα antibody is administered subcutaneously.

In some embodiments, the autoimmune disease is selected from hidradenitis suppurativa (HS), rheumatoid arthritis (RA), juvenile idiopathic arthritis, psoriatic arthritis (PsA), ankylosing spondylitis (AS), refractory asthma, Crohn's disease, psoriasis, ulcerative colitis, and uveitis.

### BRIEF DESCRIPTION OF THE DRAWINGS

Drawings are provided for illustration purposes only not for limitation.

FIGS. 1A-1B show exemplary data from the solubility study of anti-TNFα antibody 1. FIG. 1A is a graph comparing viscosity at various (50 mg/mL, 100 mg/mL, 150 mg/mL and 200 mg/mL) protein concentrations while FIG. 1B shows the monomer % change trend by SE-HPLC at the same concentrations.

FIGS. 2A-2B are exemplary graphs illustrating the antibody-dependent cellular cytotoxicity (ADCC) and complement-dependent cytotoxicity (CDC) characteristics of anti-TNFα antibody 1 and adalimumab. FIG. 2A is an exemplary graph illustrating that, in contrast to adalimumab, anti-TNFα antibody 1 does not induce ADCC FIG. 2B is an exemplary graph illustrating that CDC activity is attenuated with anti-TNFα antibody 1 compared to adalimumab.

FIG. 3 is an exemplary graph showing the neutralization of soluble recombinant human TNFα (rhTNFα) and protection in D-galactosamine-sensitized mice by anti-TNFα antibody 1 and adalimumab, with both antibodies inhibiting TNFα-included lethality in a dose-dependent manner with up to 80% survival.

FIG. 4 is an exemplary graph showing half-life of anti-TNFα antibody 1 as compared to adalimumab.

FIGS. 5A-5B show exemplary schematic diagrams. Abbreviations: SC: subcutaneous; SRC: Safety Review Committee. FIG. 5A shows a single-ascending dose study wherein each Cohort starts with a sentinel group: 1 HV dosed with anti-TNFα antibody 1 and 1 HV dosed with placebo. Following an observation period of 72 hours, if no AEs are observed that make continued dosing at that dose level inappropriate, then the remaining participants in the cohort will receive a single SC dose of anti-TNFα antibody 1 or placebo. Escalation to the next dose cohort may commence only after review of the 2-week safety data and available PK data, and approval by the SRC. This process is repeated for all subsequent cohorts. FIG. 5B. shows a multiple-ascending dose study wherein approximately 8 participants are randomly assigned 3:1 to anti-TNFα antibody 1 or placebo, per cohort. Escalation to the next dose cohort may commence only after review of the safety data by the SRC from at least 7 participants per cohort who have either completed Day 43 (Week 6) or terminated early due to a DLT or of anti-TNFα antibody 1-related AE. The decision regarding dose escalation will consider the available Safety and PK data across the study.

FIG. 6 shows simulated data for the predicted concentration of anti-TNFα antibody 1 when administered at 40 mg Q4W*3, 80 mg Q4W*3, or 120 mg Q4W*3. These doses correspond to those of the Multiple Ascending Dose study in participants with rheumatoid arthritis.

### DEFINITIONS

Antibody: The term “antibody” herein is meant a protein consisting of one or more polypeptides substantially encoded by all or part of the recognized immunoglobulin genes. The recognized immunoglobulin genes, for example in humans, include the kappa (K), lambda (l), and heavy chain genetic loci, which together comprise the myriad variable region genes, and the constant region genes mu (u), delta (d), gamma (y), sigma (s), and alpha (a) which encode the IgM, IgD, IgG (lgG1, lgG2, lgG3, and lgG4), IgE, and IgA (lgA1 and lgA2) isotypes respectively. Antibody herein is meant to include full length antibodies and antibody fragments, and may refer to a natural antibody from any organism, an engineered antibody, or an antibody generated recombinantly for experimental, therapeutic, or other purposes.

Baseline: The term “baseline” is defined as values of a parameter prior to commencement of treatment with a therapeutic. In some embodiments “baseline” is an initial measurement of a condition that is taken at an early time point and used for comparison over time to look for changes. In some embodiments, the baseline is time “zero”, before the participants in the study receive an experimental agent or intervention, or negative control; drug safety and efficacy may be determined by monitoring changes in baseline values.

Fc or Fc region: The terms “Fc” or “Fc region,” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU numbering scheme. Fc may refer to this region in isolation, or this region in the context of an Fc polypeptide, as described below. Unless indicated otherwise, the references to specific residues contained herein is according to the EU numbering scheme. The EU numbering scheme refers to the numbering of the EU antibody (Edelman et al., 1969, Proc Natl Acad Sci USA 63:78-85, hereby entirely incorporated by reference).

Modification: The term “modification” herein is meant an alteration in the physical, chemical, or sequence properties of a protein, polypeptide, antibody, or immunoglobulin. Modifications described herein include amino acid modifications and glycoform modifications.

Target Antigen: The term “target antigen” as used herein is meant the molecule that is bound by the variable region of a given antibody, or the fusion partner of an Fc fusion. A target antigen may be a protein, carbohydrate, lipid, or other chemical compound. An antibody or Fc fusion is said to be “specific” for a given target antigen based on having affinity for the target antigen. In some embodiments, the target antigen for the TNFα antibody is TNFα.

Target cell: The term “target cell” as used herein is meant a cell that expresses a target antigen.

TNFα: The term “TNFα” refers to Tumor Necrosis Factor alpha. Tumor necrosis factor (TNF) is a multifunctional cytokine that plays important roles in diverse cellular events such as cell survival, proliferation, differentiation, and death. TNF-α binds to two different receptors, which initiate signal transduction pathways. These pathways lead to various cellular responses, including cell survival, differentiation, and proliferation. In some embodiments, human TNFα comprises SEQ ID NO: 1:

(SEQ ID NO: 1)
MSTESMIRDVELAEEALPKKTGGPQGSRRCLFLSLFSFLIVAGATTLFC
LLHFGVIGPQREEFPRDLSLISPLAQAVRSSSRTPSDKPVAHVVANPQA
EGQLQWLNRRANALLANGVELRDNQLVVPSEGLYLIYSQVLFKGQGCPS
THVLLTHTISRIAVSYQTKVNLLSAIKSPCQRETPEGAEAKPWYEPIYL
GGVFQLEKGDRLSAEINRPDYLDFAESGQVYFGIIAL

### DETAILED DESCRIPTION

The present invention provides, among other things, methods of treating various autoimmune diseases by administering to a human patient in need of treatment an anti-TNFα antibody at a therapeutically effective dose and an administration interval for a treatment period sufficient to improve, stabilize or reduce one or more symptoms of the autoimmune disease relative to a control (e.g., start of treatment).

Various aspects of the invention are described in detail in the following sections. The use of sections is not meant to limit the invention. Each section can apply to any aspect of the invention. In this application, the use of “or” means “and/or” unless stated otherwise.

### Anti-TNFα Antibody 1 and Variants Thereof

According to the present invention, anti-TNFα antibody 1 and a variant thereof is used to treat a human patient suffering from an autoimmune disease. Anti-TNFα antibody 1 is a monoclonal antibody specific for TNFα comprising: a light chain comprising a variable region having:
a CDR1 comprising RASQGIRNYLA (SEQ ID NO: 2),
a CDR2 comprising AASTLOS (SEQ ID NO: 3), and
a CDR3 comprising QRYNRAPYT (SEQ ID NO: 4); and
a heavy chain comprising a variable region having
a CDR1 comprising DYAMH (SEQ ID NO: 5),
a CDR2 comprising AITWNSGHIDYADSVEG (SEQ ID NO: 6), and
a CDR3 comprising VSYLSTASSLDY (SEQ ID NO: 7),
wherein the heavy chain comprises amino acid substitutions in the Fc region M428L and N434S as compared to SEQ ID NO: 8:

(SEQ ID NO: 8)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPPVAGPSVFLFPPKPKDTLMISRTPEVTCVVVD
VSHEDPEVQFNWYVDGVEVHNAKTKPREEQFNSTFRVVSVLTVVHQDWL
NGKEYKCKVSNKGLPAPIEKTISKTKGQPREPQVYTLPPSREEMTKNQV
SLTCLVKGFYPSDIAVEWESNGOPENNYKTTPPMLDSDGSFFLYSKLTV
DKSRWQQGNVFSCSVLHEALHSHYTQKSLSLSPGK,

wherein the numbering is according to the EU index, as in Kabat.

In some embodiments, anti-TNFα antibody 1 comprises: a light chain comprising an amino acid sequence of (SEQ ID NO: 9) and heavy chain comprising an amino acid sequence of, (SEQ ID NO: 10), as given by Table 1.

TABLE 1
Sequence of heavy chain and light chain amino acid sequence of anti-TNFα
antibody 1.

| Heavy Chain | Light Chain |
|---|---|
| EVQLVESGGGLVQPGRSLRLSCAASGFTF | DIQMTQSPSSLSASVGDRVTITCRASQGIRNY |
| DDYAMHWVRQAPGKGLEWVSAITWNSGHI | LAWYQQKPGKAPKLLIYAASTLQSGVPSRFSG |
| DYADSVEGRFTISRDNAKNSLYLQMNSLR | SGSGTDFTLTISSLQPEDVATYYCQRYNRAPY |
| AEDTAVYYCAKVSYLSTASSLDYWGQGTL | TFGQGTKVEIKRTVAAPSVFIFPPSDEQLKSG |
| VTVSSASTKGPSVFPLAPSSKSTSGGTAA | TAS (SEQ ID NO: 9) |
| LGCLVKDYFPEPVTVSWNSGALTSGVHTF | |
| PAVLQSSGLYSLSSVVTVPSSSLGTQTYI | |
| CNVNHKPSNTKVDKKVEPKSCDKTHTCPP | |
| CPAPPVAGPSVFLFPPKPKDTLMISRTPE | |
| VTCVVVDVSHEDPEVQFNWYVDGVEVHNA | |
| KTKPREEQFNSTFRVVSVLTVVHQDWLNG | |
| KEYKCKVSNKGLPAPIEKTISKTKGQPRE | |
| PQVYTLPPSREEMTKNQVSLTCLVKGFYP | |
| SDIAVEWESNGQPENNYKTTPPMLDSDGS | |
| FFLYSKLTVDKSRWQQGNVFSCSVLHEAL | |
| HSHYTQKSLSLSPGK (SEQ ID NO: 10) | |

Variable region

| | |
|---|---|
| EVQLVESGGGLVQPGRSLRLSCAASGFTF | DIQMTQSPSSLSASVGDRVTITCRASQGIRNY |
| DDYAMHWVRQAPGKGLEWVSAITWNSGHI | LAWYQQKPGKAPKLLIYAASTLQSGVPSRFSG |
| DYADSVEGRFTISRDNAKNSLYLQMNSLR | SGSGTDFTLTISSLQPEDVATYYCQRYNRAPY |
| AEDTAVYYCAKVSYLSTASSLDYWGQGTL | TFGQGTKVEIK (SEQ ID NO: 11) |
| VTVSS (SEQ ID NO: 12) | |

In some embodiments, anti-TNFα antibody 1 comprises a light chain variable region comprising SEQ ID NO: 11 and a heavy chain variable region comprising SEQ ID NO: 12.

In some embodiments, an anti-TNFα antibody has a pI of between 7.0-9.0. In some embodiments, an anti-TNFα antibody has a pI of between 7.8-8.8. In some embodiments, an anti-TNFα antibody has a pI of between 8.0-8.5. In some embodiments, an anti-TNFα antibody has a pI of 8.3.

### Variants

In some embodiments, the anti-TNFα antibody comprises a variant of anti-TNFα antibody 1.

In some embodiments, the anti-TNFα antibody comprises a heavy chain variable region (VH) and/or light chain variable region (VL), comprising a CDR1, a CDR2, a CDR3, each which differs by no more than 5 amino acid residues from each of SEQ ID NO: 2, SEQ ID NO: 3, SEQ ID NO: 4, SEQ ID NO: 5, SEQ ID NO: 6, and/or SEQ ID NO: 7. In some embodiments, the anti-TNFα antibody comprises a heavy chain variable region (VH) and/or light chain variable region (VL), comprising a CDR1, a CDR2, a CDR3, each which differs by no more than 4 amino acid residues from each of SEQ ID NO: 2, SEQ ID NO: 3, SEQ ID NO: 4, SEQ ID NO: 5, SEQ ID NO: 6, and/or SEQ ID NO: 7. In some embodiments, the anti-TNFα antibody comprises a heavy chain variable region (VH) and/or light chain variable region (VL), comprising a CDR1, a CDR2, a CDR3, each which differs by no more than 3 amino acid residues from each of SEQ ID NO: 2, SEQ ID NO: 3, SEQ ID NO: 4, SEQ ID NO: 5, SEQ ID NO: 6, and/or SEQ ID NO: 7. In some embodiments, the anti-TNFα antibody comprises a heavy chain variable region (VH) and/or light chain variable region (VL), comprising a CDR1, a CDR2, a CDR3, each which differs by no more than 2 amino acid residues from each of SEQ ID NO: 2, SEQ ID NO: 3, SEQ ID NO: 4, SEQ ID NO: 5, SEQ ID NO: 6, and/or SEQ ID NO: 7. In some embodiments, the anti-TNFα antibody comprises a heavy chain variable region (VH) and/or light chain variable region (VL), comprising a CDR1, a CDR2, a CDR3, each which differs by no more than 1 amino acid residue from each of SEQ ID NO: 2, SEQ ID NO: 3, SEQ ID NO: 4, SEQ ID NO: 5, SEQ ID NO: 6, and/or SEQ ID NO: 7.

In some embodiments, the anti-TNFα antibody comprises a light chain that is at least 80% identical to SEQ ID NO: 9. In some embodiments, the anti-TNFα antibody comprises a light chain that is at least 85% identical to SEQ ID NO: 9. In some embodiments, the anti-TNFα antibody comprises a light chain that is at least 90% identical to SEQ ID NO: 9. In some embodiments, the anti-TNFα antibody comprises a light chain that is at least 91% identical to SEQ ID NO: 9. In some embodiments, the anti-TNFα antibody comprises a light chain that is at least 92% identical to SEQ ID NO: 9. In some embodiments, the anti-TNFα antibody comprises a light chain that is at least 92% identical to SEQ ID NO: 9. In some embodiments, the anti-TNFα antibody comprises a light chain that is at least 93% identical to SEQ ID NO: 9. In some embodiments, the anti-TNFα antibody comprises a light chain that is at least 94% identical to SEQ ID NO: 9. In some embodiments, the anti-TNFα antibody comprises a light chain that is at least 95% identical to SEQ ID NO: 9. In some embodiments, the anti-TNFα antibody comprises a light chain that is at least 96% identical to SEQ ID NO: 9. In some embodiments, the anti-TNFα antibody comprises a light chain that is at least 97% identical to SEQ ID NO: 9. In some embodiments, the anti-TNFα antibody comprises a light chain that is at least 98% identical to SEQ ID NO: 9. In some embodiments, the anti-TNFα antibody comprises a light chain that is at least 99% identical to SEQ ID NO: 9.

In some embodiments, the anti-TNFα antibody comprises a heavy chain that is at least 80% identical to SEQ ID NO: 10 and further comprises an Fc region comprising M428L and N434S substitutions. In some embodiments, the anti-TNFα antibody comprises a heavy chain that is at least 85% identical to SEQ ID NO: 10 and further comprises an Fc region comprising M428L and N434S substitutions. In some embodiments, the anti-TNFα antibody comprises a heavy chain that is at least 90% identical to SEQ ID NO: 10 and further comprises an Fc region comprising M428L and N434S substitutions. In some embodiments, the anti-TNFα antibody comprises a heavy chain that is at least 91% identical to SEQ ID NO: 10 and further comprises an Fc region comprising M428L and N434S substitutions. In some embodiments, the anti-TNFα antibody comprises a heavy chain that is at least 92% identical to SEQ ID NO: 10 and further comprises an Fc region comprising M428L and N434S substitutions. In some embodiments, the anti-TNFα antibody comprises a heavy chain that is at least 92% identical to SEQ ID NO: 10 and further comprises an Fc region comprising M428L and N434S substitutions. In some embodiments, the anti-TNFα antibody comprises a heavy chain that is at least 93% identical to SEQ ID NO: 10 and further comprises an Fc region comprising M428L and N434S substitutions. In some embodiments, the anti-TNFα antibody comprises a heavy chain that is at least 94% identical to SEQ ID NO: 10 and further comprises an Fc region comprising M428L and N434S substitutions. In some embodiments, the anti-TNFα antibody comprises a heavy chain that is at least 95% identical to SEQ ID NO: 10 and further comprises an Fc region comprising M428L and N434S substitutions. In some embodiments, the anti-TNFα antibody comprises a heavy chain that is at least 96% identical to SEQ ID NO: 10 and further comprises an Fc region comprising M428L and N434S substitutions. In some embodiments, the anti-TNFα antibody comprises a heavy chain that is at least 97% identical to SEQ ID NO: 10 and further comprises an Fc region comprising M428L and N434S substitutions. In some embodiments, the anti-TNFα antibody comprises a heavy chain that is at least 98% identical to SEQ ID NO: 10 and further comprises an Fc region comprising M428L and N434S substitutions. In some embodiments, the anti-TNFα antibody comprises a heavy chain that is at least 99% identical to SEQ ID NO: 10 and further comprises an Fc region comprising M428L and N434S substitutions.

In some embodiments, the anti-TNFα antibody comprises a light chain variable region that is at least 80% identical to SEQ ID NO: 11. In some embodiments, the anti-TNFα antibody comprises a light chain variable region that is at least 85% identical to SEQ ID NO: 11. In some embodiments, the anti-TNFα antibody comprises a light chain variable region that is at least 90% identical to SEQ ID NO: 11. In some embodiments, the anti-TNFα antibody comprises a light chain variable region that is at least 91% identical to SEQ ID NO: 11. In some embodiments, the anti-TNFα antibody comprises a light chain variable region that is at least 92% identical to SEQ ID NO: 11. In some embodiments, the anti-TNFα antibody comprises a light chain variable region that is at least 92% identical to SEQ ID NO: 11. In some embodiments, the anti-TNFα antibody comprises a light chain variable region that is at least 93% identical to SEQ ID NO: 11. In some embodiments, the anti-TNFα antibody comprises a light chain variable region that is at least 94% identical to SEQ ID NO: 11. In some embodiments, the anti-TNFα antibody comprises a light chain variable region that is at least 95% identical to SEQ ID NO: 11. In some embodiments, the anti-TNFα antibody comprises a light chain variable region that is at least 96% identical to SEQ ID NO: 11. In some embodiments, the anti-TNFα antibody comprises a light chain variable region that is at least 97% identical to SEQ ID NO: 11. In some embodiments, the anti-TNFα antibody comprises a light chain variable region that is at least 98% identical to SEQ ID NO: 11. In some embodiments, the anti-TNFα antibody comprises a light chain variable region that is at least 99% identical to SEQ ID NO: 11.

In some embodiments, the anti-TNFα antibody comprises a heavy chain variable region that is at least 80% identical to SEQ ID NO: 11. In some embodiments, the anti-TNFα antibody comprises a heavy chain variable region that is at least 85% identical to SEQ ID NO: 11. In some embodiments, the anti-TNFα antibody comprises a heavy chain variable region that is at least 90% identical to SEQ ID NO: 11. In some embodiments, the anti-TNFα antibody comprises a heavy chain variable region that is at least 91% identical to SEQ ID NO: 11. In some embodiments, the anti-TNFα antibody comprises a heavy chain variable region that is at least 92% identical to SEQ ID NO: 11. In some embodiments, the anti-TNFα antibody comprises a heavy chain variable region that is at least 92% identical to SEQ ID NO: 11. In some embodiments, the anti-TNFα antibody comprises a heavy chain variable region that is at least 93% identical to SEQ ID NO: 11. In some embodiments, the anti-TNFα antibody comprises a heavy chain variable region that is at least 94% identical to SEQ ID NO: 11. In some embodiments, the anti-TNFα antibody comprises a heavy chain variable region that is at least 95% identical to SEQ ID NO: 11. In some embodiments, the anti-TNFα antibody comprises a heavy chain variable region that is at least 96% identical to SEQ ID NO: 11. In some embodiments, the anti-TNFα antibody comprises a heavy chain variable region that is at least 97% identical to SEQ ID NO: 11. In some embodiments, the anti-TNFα antibody comprises a heavy chain variable region that is at least 98% identical to SEQ ID NO: 11. In some embodiments, the anti-TNFα antibody comprises a heavy chain variable region that is at least 99% identical to SEQ ID NO: 11.

In some embodiments, the anti-TNFα antibody comprises a heavy chain variable region that is at least 80% identical to SEQ ID NO: 12. In some embodiments, the anti-TNFα antibody comprises a heavy chain variable region that is at least 85% identical to SEQ ID NO: 12. In some embodiments, the anti-TNFα antibody comprises a heavy chain variable region that is at least 90% identical to SEQ ID NO: 12. In some embodiments, the anti-TNFα antibody comprises a heavy chain variable region that is at least 91% identical to SEQ ID NO: 12. In some embodiments, the anti-TNFα antibody comprises a heavy chain variable region that is at least 92% identical to SEQ ID NO: 12. In some embodiments, the anti-TNFα antibody comprises a heavy chain variable region that is at least 92% identical to SEQ ID NO: 12. In some embodiments, the anti-TNFα antibody comprises a heavy chain variable region that is at least 93% identical to SEQ ID NO: 12. In some embodiments, the anti-TNFα antibody comprises a heavy chain variable region that is at least 94% identical to SEQ ID NO: 12. In some embodiments, the anti-TNFα antibody comprises a heavy chain variable region that is at least 95% identical to SEQ ID NO: 12. In some embodiments, the anti-TNFα antibody comprises a heavy chain variable region that is at least 96% identical to SEQ ID NO: 12. In some embodiments, the anti-TNFα antibody comprises a heavy chain variable region that is at least 97% identical to SEQ ID NO: 12. In some embodiments, the anti-TNFα antibody comprises a heavy chain variable region that is at least 98% identical to SEQ ID NO: 12. In some embodiments, the anti-TNFα antibody comprises a heavy chain variable region that is at least 99% identical to SEQ ID NO: 12.

### Formulations

Formulating high concentration antibodies presents significant challenges due to various factors. High concentration monoclonal antibody solutions can lead to viscosity increases, solubility limitations like gelation or phase separation, and physical stability challenges such as opalescence. These challenges are particularly prevalent in high concentration antibody products (HCAPs) with protein concentrations of ≥50 mg/mL, requiring robust processes for formulation, stabilization, development, and manufacturing. Moreover, high concentration antibody preparations can undergo hydrolytically driven chemical degradations like deamidation, isomerization, and cleavage of peptide bonds, leading to irreversible covalent aggregates that may affect safety and efficacy. To mitigate these risks, formulating high concentration antibody products requires addressing challenges related to physical stability, solubility, viscosity, aggregation, and immunogenicity.

Salt and amino acids are frequently used at high concentration (>100 mM) to maintain stability and prevent aggregation of high-concentration antibody formulation. However, formulations with high salt content or specific amino acids may raise immunogenicity concerns, potentially triggering immune responses in patients receiving the antibody products. Additionally, high salt concentrations may contribute to increased viscosity in antibody formulations, which can pose challenges during manufacturing processes, such as elevated shear stress during pumping and membrane clogging. In some embodiments, the present invention is based on the discovery of a stable formulation comprising an anti-TNFα antibody at a concentration greater than (e.g., >50 mg/mL) with low concentration of salt or amino acids (e.g., <100 mM) while maintaining low viscosity (e.g., 3.2 mPa*s) and stability.

The concentration of the aqueous formulation of the invention is not limited by the protein size and the formulation may include any size range of proteins. Included within the scope of the invention is an aqueous formulation comprising at least 50 mg/mL and as much as 200 mg/mL or more of a protein, which may range in size from 5 kDa to 150 kDa or more. In one embodiment, the protein in the formulation of the invention is at least 15 kD in size, at least 20 kD in size; at least 47 kD in size; at least 60 kD in size; at least 80 kD in size; at least 100 kD in size; at least 120 kD in size; at least 140 kD in size; at least 160 kD in size; or greater than 160 kD in size. Ranges intermediate to the above recited sizes are also intended to be part of this invention. In addition, ranges of values using a combination of any of the above recited values as upper and/or lower limits are intended to be included.

In some embodiments, the anti-TNFα antibody included in the stable formulation of the invention has a given concentration, including, for example, a concentration of at least 1 mg/mL, at least 10 mg/mL, at least 50 mg/mL, at least 100 mg/mL, at least 150 mg/mL, at least 200 mg/mL, at least 240 mg/mL, at least 250 mg/mL, or greater than 250 mg/mL.

In some embodiments, the stable formulation of the present invention comprises an anti-TNFα antibody at a concentration between 50 mg/mL and 250 mg/mL. In some embodiments, the stable formulation of the present invention comprises an anti-TNFα antibody at a concentration between 75 mg/mL and 225 mg/mL. In some embodiments, the stable formulation of the present invention comprises an anti-TNFα antibody at a concentration between 50 mg/ml and 250 mg/mL. In some embodiments, the stable formulation of the present invention comprises an anti-TNFα antibody at a concentration between 100 mg/mL and 200 mg/mL. In some embodiments, the stable formulation of the present invention comprises an anti-TNFα antibody at a concentration between 125 mg/mL and 175 mg/mL. In some embodiments, the stable formulation of the present invention comprises an anti-TNFα antibody at a concentration between 135 mg/mL and 165 mg/mL.

In some embodiments, the stable formulation of the present invention comprises an anti-TNFα antibody at a concentration between 65 mg/mL and 135 mg/mL. In some embodiments, the stable formulation of the present invention comprises an anti-TNFα antibody at a concentration between 85 mg/mL and 115 mg/mL. In some embodiments, the stable formulation of the present invention comprises an anti-TNFα antibody at a concentration between 90 mg/mL and 110 mg/mL.

In some embodiments, the stable formulation comprises an anti-TNFα antibody at a concentration of at least 50 mg/mL. In some embodiments, the stable formulation comprises an anti-TNFα antibody at a concentration of at least 60 mg/mL. In some embodiments, the stable formulation comprises an anti-TNFα antibody at a concentration of at least 70 mg/mL. In some embodiments, the stable formulation comprises an anti-TNFα antibody at a concentration of at least 80 mg/mL. In some embodiments, the stable formulation comprises an anti-TNFα antibody at a concentration of at least 90 mg/mL. In some embodiments, the stable formulation comprises an anti-TNFα antibody at a concentration of at least 90 mg/mL. In some embodiments, the stable formulation comprises an anti-TNFα antibody at a concentration of at least 100 mg/mL. In some embodiments, the stable formulation comprises an anti-TNFα antibody at a concentration of at least 110 mg/mL. In some embodiments, the stable formulation comprises an anti-TNFα antibody at a concentration of at least 120 mg/mL. In some embodiments, the stable formulation comprises an anti-TNFα antibody at a concentration of at least 130 mg/mL. In some embodiments, the stable formulation comprises an anti-TNFα antibody at a concentration of at least 140 mg/mL. In some embodiments, the stable formulation comprises an anti-TNFα antibody at a concentration of at least 150 mg/mL. In some embodiments, the stable formulation comprises an anti-TNFα antibody at a concentration of at least 160 mg/mL. In some embodiments, the stable formulation comprises an anti-TNFα antibody at a concentration of at least 170 mg/mL. In some embodiments, the stable formulation comprises an anti-TNFα antibody at a concentration of at least 180 mg/mL. In some embodiments, the stable formulation comprises an anti-TNFα antibody at a concentration of at least 190 mg/mL. In some embodiments, the stable formulation comprises an anti-TNFα antibody at a concentration of at least 200 mg/mL. In some embodiments, the stable formulation comprises an anti-TNFα antibody at a concentration of at least 210 mg/mL. In some embodiments, the stable formulation comprises an anti-TNFα antibody at a concentration of at least 220 mg/mL. In some embodiments, the stable formulation comprises an anti-TNFα antibody at a concentration of at least 230 mg/mL. In some embodiments, the stable formulation comprises an anti-TNFα antibody at a concentration of at least 240 mg/mL. In some embodiments, the stable formulation comprises an anti-TNFα antibody at a concentration of at least 250 mg/mL.

### Buffer

In some embodiments of the invention, a formulation comprises a buffer to control pH. Suitable buffers generally include, for example, acetate, citrate, histidine, phosphate, succinate, tris (hydroxymethyl) aminomethane (“Tris”) and other organic acids. In some embodiments, the formulation comprises a buffer selected from citrate, histidine, phosphate and succinate. In some embodiments, the formulation comprises a buffer at a concentration ranging from 5 mM to 100 mM. In some embodiments, the formulation comprises a buffer at a concentration ranging from 5 mM to 75 mM. In some embodiments, the formulation comprises a buffer at a concentration ranging from 5 mM to 50 mM. In some embodiments, the formulation comprises a buffer at a concentration ranging from 5 mM to 40 mM. In some embodiments, the formulation comprises a buffer at a concentration ranging from 5 mM to 30 mM. In some embodiments, the formulation comprises a buffer at a concentration ranging from 5 mM to 20 mM. In some embodiments, the formulation comprises a buffer at a concentration ranging from 5 mM to 10 mM. In some embodiments, the formulation comprises a buffer at a concentration ranging from 10 mM to 100 mM. In some embodiments, the formulation comprises a buffer at a concentration ranging from 20 mM to 100 mM. In some embodiments, the formulation comprises a buffer at a concentration ranging from 30 mM to 100 mM. In some embodiments, the formulation comprises a buffer at a concentration ranging from 40 mM to 100 mM. In some embodiments, the formulation comprises a buffer at a concentration ranging from 50 mM to 100 mM. In some embodiments, the formulation comprises a buffer at a concentration ranging from 75 mM to 100 mM. In some embodiments, the formulation comprises a buffer at a concentration of 10 mM. In some embodiments, the formulation comprises a buffer at a concentration of less than 60 mM. In some embodiments, the formulation comprises a buffer at a concentration of less than 55 mM. In some embodiments, the formulation comprises a buffer at a concentration of less than 50 mM. In some embodiments, the formulation comprises a buffer at a concentration of less than 45 mM. In some embodiments, the formulation comprises a buffer at a concentration of less than 40 mM. In some embodiments, the formulation comprises a buffer at a concentration of less than 35 mM. In some embodiments, the formulation comprises a buffer at a concentration of less than 30 mM. In some embodiments, the formulation comprises a buffer at a concentration of less than 25 mM. In some embodiments, the formulation comprises a buffer at a concentration of 5 mM. In some embodiments, the formulation comprises a buffer at a concentration of 10 mM. In some embodiments, the formulation comprises a buffer at a concentration of 15 mM. In some embodiments, the formulation comprises a buffer at a concentration of 20 mM. In some embodiments, the formulation comprises a buffer at a concentration of 25 mM. In some embodiments, the formulation comprises a buffer at a concentration of 30 mM. In some embodiments, the formulation comprises a buffer at a concentration of 35 mM. In some embodiments, the formulation comprises a buffer at a concentration of 40 mM.

In some embodiments, the formulation comprises a buffer comprising acetate. In some embodiments, the formulation comprises acetate at a concentration ranging from 5 mM to 50 mM. In some embodiments, the formulation comprises acetate at a concentration ranging from 5 mM to 40 mM. In some embodiments, the formulation comprises acetate at a concentration ranging from 5 mM to 30 mM. In some embodiments, the formulation comprises acetate at a concentration ranging from 5 mM to 20 mM. In some embodiments, the formulation comprises acetate at a concentration ranging from 5 mM to 10 mM. In some embodiments, the formulation comprises acetate at a concentration ranging from 10 mM to 50 mM. In some embodiments, the formulation comprises acetate at a concentration ranging from 20 mM to 50 mM. In some embodiments, the formulation comprises acetate at a concentration ranging from 30 mM to 50 mM. In some embodiments, the formulation comprises acetate at a concentration ranging from 40 mM to 50 mM. In some embodiments, the formulation comprises acetate at a concentration of less than 60 mM. In some embodiments, the formulation comprises acetate at a concentration of less than 55 mM. In some embodiments, the formulation comprises acetate at a concentration of less than 50 mM. In some embodiments, the formulation comprises acetate at a concentration of less than 45 mM. In some embodiments, the formulation comprises acetate at a concentration of less than 40 mM. In some embodiments, the formulation comprises acetate at a concentration of less than 35 mM. In some embodiments, the formulation comprises acetate at a concentration of less than 30 mM. In some embodiments, the formulation comprises acetate at a concentration of less than 25 mM. In some embodiments, the formulation comprises acetate at a concentration of 5 mM. In some embodiments, the formulation comprises acetate at a concentration of 10 mM. In some embodiments, the formulation comprises acetate at a concentration of 15 mM. In some embodiments, the formulation comprises acetate at a concentration of 20 mM. In some embodiments, the formulation comprises acetate at a concentration of 25 mM. In some embodiments, the formulation comprises acetate at a concentration of 30 mM. In some embodiments, the formulation comprises acetate at a concentration of 35 mM. In some embodiments, the formulation comprises acetate at a concentration of 40 mM.

In some embodiments, the formulation comprises a buffer comprising a histidine buffer. In some embodiments, the histidine buffer comprises L-histidine. In some embodiments, the formulation comprises histidine buffer at a concentration ranging from 5 mM to 50 mM. In some embodiments, the formulation comprises histidine buffer at a concentration ranging from 5 mM to 40 mM. In some embodiments, the formulation comprises histidine buffer at a concentration ranging from 5 mM to 30 mM. In some embodiments, the formulation comprises histidine buffer at a concentration ranging from 5 mM to 20 mM. In some embodiments, the formulation comprises histidine buffer at a concentration ranging from 5 mM to 10 mM. In some embodiments, the formulation comprises histidine buffer at a concentration ranging from 10 mM to 50 mM. In some embodiments, the formulation comprises histidine buffer at a concentration ranging from 20 mM to 50 mM. In some embodiments, the formulation comprises histidine buffer at a concentration ranging from 30 mM to 50 mM. In some embodiments, the formulation comprises histidine buffer at a concentration ranging from 40 mM to 50 mM. In some embodiments, the formulation comprises histidine buffer at a concentration of less than 60 mM. In some embodiments, the formulation comprises histidine buffer at a concentration of less than 55 mM. In some embodiments, the formulation comprises histidine buffer at a concentration of less than 50 mM. In some embodiments, the formulation comprises histidine buffer at a concentration of less than 45 mM. In some embodiments, the formulation comprises histidine buffer at a concentration of less than 40 mM. In some embodiments, the formulation comprises histidine buffer at a concentration of less than 35 mM. In some embodiments, the formulation comprises histidine buffer at a concentration of less than 30 mM. In some embodiments, the formulation comprises histidine buffer at a concentration of less than 25 mM. In some embodiments, the formulation comprises histidine buffer at a concentration of 5 mM. In some embodiments, the formulation comprises histidine buffer at a concentration of 10 mM. In some embodiments, the formulation comprises histidine buffer at a concentration of 15 mM. In some embodiments, the formulation comprises histidine buffer at a concentration of 20 mM. In some embodiments, the formulation comprises histidine buffer at a concentration of 25 mM. In some embodiments, the formulation comprises histidine buffer at a concentration of 30 mM. In some embodiments, the formulation comprises histidine buffer at a concentration of 35 mM. In some embodiments, the formulation comprises histidine buffer at a concentration of 40 mM.

### Surfactant

In some embodiments of the invention, a formulation further comprises a surfactant. Exemplary surfactants include nonionic surfactants such as polysorbates (e.g., polysorbate 20, polysorbate 40, or polysorbate 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; and the MONAQUAT™ series (Mona Industries, Inc., Paterson, N.J.), polyethylene glycol (PEG), polypropylene glycol, and copolymers of ethylene and propylene glycol (e.g., Pluronics, PF68, etc.). Typically, the amount of surfactant added is such that it reduces aggregation of the antibody and minimizes the formation of particulates. For example, a surfactant may be present in a formulation at a concentration from 0.001 to 0.5% (w/v) (e.g., 0.075%). In particular, a surfactant may be present in a formulation at a concentration of 0.005% (w/v), 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), etc. In some embodiments, a surfactant may be present in a formulation at a concentration of 0.02% (w/v). In some embodiments, a surfactant may be present in a formulation at a concentration of 0.04% (w/v). In some embodiments, a surfactant may be present in a formulation at a concentration of 0.6% (w/v).

In some embodiments, the surfactant comprises polysorbate 80 (PS 80). In some embodiments, polysorbate 80 (PS80) is present a concentration of between 0.001 (w/v/) and 0.1% (w/v). In some embodiments, polysorbate 80 (PS80) is present at a concentration of between 0.01% (w/v) and 0.6% (w/v). In some embodiments, polysorbate 80 (PS80) is present at a concentration of 0.01% (w/v), 0.02% (w/v), 0.03% (w/v/), 0.04% (w/v), 0.05% (w/v), 0.06% (w/v), 0.07% (w/v), 0.08% (w/v), 0.09% (w/v), 0.1% (w/v). In some embodiments, polysorbate 80 (PS80) is present at a concentration of 0.01% (w/v). In some embodiments, polysorbate 80 (PS80) is present at a concentration of 0.02% (w/v). In some embodiments, polysorbate 80 (PS80) is present at a concentration of 0.03% (w/v). In some embodiments, polysorbate 80 (PS80) is present at a concentration of 0.04% (w/v). In some embodiments, polysorbate 80 (PS80) is present at a concentration of 0.05% (w/v). In some embodiments, polysorbate 80 (PS80) is present at a concentration of 0.06% (w/v). In some embodiments, polysorbate 80 (PS80) is present at a concentration of 0.07% (w/v). In some embodiments, polysorbate 80 (PS80) is present at a concentration of 0.08% (w/v). In some embodiments, polysorbate 80 (PS80) is present at a concentration of 0.09% (w/v). In some embodiments, polysorbate 80 (PS80) is present at a concentration of 0.1% (w/v). Alternatively, or in addition, the surfactant may be added to a lyophilized formulation, pre-lyophilized formulation and/or a reconstituted formulation.

### Excipients

In some embodiments of the invention, the stable formulation further comprises one or more excipients. In some embodiments, the stable formulation further compromises one or more of a sugar, a polyol, a salt, and an amino acid. In some embodiments, the stable formulation further comprises a sugar. In some embodiments, the stable formulation further comprises a polyol. In some embodiments, the stable formulation further comprises a salt. In some embodiments, the stable formulation further comprises an amino acid. In some embodiments, the stable formulation comprises a sugar and a salt. In some embodiments, the stable formulation comprises a sugar and an amino acid.

### Sugars

In some embodiments, the stable formulation further comprises a sugar. Suitable sugars include monosaccharides, disaccharides, and/or polysaccharides. In some embodiments, the sugar comprises sucrose, trehalose, fructose, maltose, galactose, glucose, D-mannose, sorbose, lactose, cellobiose, raffinose, melezitose, maltodextrins, dextrans, or starches. In some embodiments, the sugar comprises sucrose. In some embodiments, the sugar comprises trehalose.

In some embodiments, the stable formulation comprises a sugar at a concentration of between 0.1% (w/v) and 15% (w/v). In some embodiments, the sugar is present at a concentration of between 1% (w/v) and 10% (w/v). In some embodiments, the sugar is present at a concentration of 4% (w/v) and 8% (w/v). In some embodiments, the sugar is present at a concentration of 1% (w/v). In some embodiments, the sugar is present at a concentration of 1.5% (w/v). In some embodiments, the sugar is present at a concentration of 2% (w/v). In some embodiments, the sugar is present at a concentration of 2.5% (w/v). In some embodiments, the sugar is present at a concentration of 3% (w/v). In some embodiments, the sugar is present at a concentration of 3.5% (w/v). In some embodiments, the sugar is present at a concentration of 4% (w/v). In some embodiments, the sugar is present at a concentration of 4.5% (w/v). In some embodiments, the sugar is present at a concentration of 5% (w/v). In some embodiments, the sugar is present at a concentration of 5.5% (w/v). In some embodiments, the sugar is present at a concentration of 6% (w/v). In some embodiments, the sugar is present at a concentration of 6.5% (w/v). In some embodiments, the sugar is present at a concentration of 7% (w/v). In some embodiments, the sugar is present at a concentration of 7.5% (w/v). In some embodiments, the sugar is present at a concentration of 8% (w/v). In some embodiments, the sugar is present at a concentration of 8.5% (w/v). In some embodiments, the sugar is present at a concentration of 9% (w/v). In some embodiments, the sugar is present at a concentration of 9.5% (w/v). In some embodiments, the sugar is present at a concentration of 10% (w/v). In some embodiments, the sugar is present at a concentration of 10.5% (w/v). In some embodiments, the sugar is present at a concentration of 11% (w/v). In some embodiments, the sugar is present at a concentration of 11.5% (w/v), 12% (w/v). In some embodiments, the sugar is present at a concentration of 12.5% (w/v). In some embodiments, the sugar is present at a concentration of 13% (w/v). In some embodiments, the sugar is present at a concentration of 13.5% (w/v). In some embodiments, the sugar is present at a concentration of 14% (w/v). In some embodiments, the sugar is present at a concentration of 14.5% (w/v). In some embodiments, the sugar is present at a concentration of 15% (w/v).

In some embodiments, the sugar comprises sucrose. In some embodiments, sucrose is present at a concentration of between 0.1% (w/v) and 15% (w/v). In some embodiments, sucrose is present at a concentration of between 1% (w/v) and 10% (w/v). In some embodiments, sucrose is present at a concentration of between 4% (w/v) and 8% (w/v). In some embodiments, sucrose is present at a concentration of 1% (w/v). In some embodiments, sucrose is present at a concentration of 1.5% (w/v). In some embodiments, sucrose is present at a concentration of 2% (w/v). In some embodiments, sucrose is present at a concentration of 2.5% (w/v). In some embodiments, sucrose is present at a concentration of 3% (w/v). In some embodiments, sucrose is present at a concentration of 3.5% (w/v). In some embodiments, sucrose is present at a concentration of 4% (w/v). In some embodiments, sucrose is present at a concentration of 4.5% (w/v). In some embodiments, sucrose is present at a concentration of 5% (w/v). In some embodiments, sucrose is present at a concentration of 5.5% (w/v). In some embodiments, sucrose is present at a concentration of 6% (w/v). In some embodiments, sucrose is present at a concentration of 6.5% (w/v). In some embodiments, sucrose is present at a concentration of 7% (w/v). In some embodiments, sucrose is present at a concentration of 7.5% (w/v). In some embodiments, sucrose is present at a concentration of 8% (w/v). In some embodiments, sucrose is present at a concentration of 8.5% (w/v). In some embodiments, sucrose is present at a concentration of 9% (w/v). In some embodiments, sucrose is present at a concentration of 9.5% (w/v). In some embodiments, sucrose is present at a concentration of 10% (w/v). In some embodiments, sucrose is present at a concentration of 10.5% (w/v). In some embodiments, sucrose is present at a concentration of 11% (w/v). In some embodiments, sucrose is present at a concentration of 11.5% (w/v). In some embodiments, sucrose is present at a concentration of 12% (w/v). In some embodiments, sucrose is present at a concentration of 12.5% (w/v). In some embodiments, sucrose is present at a concentration of 13% (w/v). In some embodiments, sucrose is present at a concentration of 13.5% (w/v). In some embodiments, sucrose is present at a concentration of 14% (w/v). In some embodiments, sucrose is present at a concentration of 14.5% (w/v). In some embodiments, sucrose is present at a concentration of 15% (w/v).

In some embodiments, the sugar comprises trehalose. In some embodiments, trehalose is present at a concentration of between 0.1% (w/v) and 15% (w/v). In some embodiments, trehalose is present at a concentration of between 1% (w/v) and 10% (w/v). In some embodiments, trehalose is present at a concentration of 4% (w/v) and 8% (w/v). In some embodiments, trehalose is present at a concentration of 1% (w/v). In some embodiments, trehalose is present at a concentration of 1.5% (w/v). In some embodiments, trehalose is present at a concentration of 2% (w/v). In some embodiments, trehalose is present at a concentration of 2.5% (w/v). In some embodiments, trehalose is present at a concentration of 3% (w/v). In some embodiments, trehalose is present at a concentration of 3.5% (w/v). In some embodiments, trehalose is present at a concentration of 4% (w/v). In some embodiments, trehalose is present at a concentration of 4.5% (w/v). In some embodiments, trehalose is present at a concentration of 5% (w/v). In some embodiments, trehalose is present at a concentration of 5.5% (w/v). In some embodiments, trehalose is present at a concentration of 6% (w/v). In some embodiments, trehalose is present at a concentration of 6.5% (w/v). In some embodiments, trehalose is present at a concentration of 7% (w/v). In some embodiments, trehalose is present at a concentration of 7.5% (w/v). In some embodiments, trehalose is present at a concentration of 8% (w/v). In some embodiments, trehalose is present at a concentration of 8.5% (w/v). In some embodiments, trehalose is present at a concentration of 9% (w/v). In some embodiments, trehalose is present at a concentration of 9.5% (w/v). In some embodiments, trehalose is present at a concentration of 10% (w/v). In some embodiments, trehalose is present at a concentration of 10.5% (w/v). In some embodiments, trehalose is present at a concentration of 11% (w/v). In some embodiments, trehalose is present at a concentration of 11.5% (w/v). In some embodiments, trehalose is present at a concentration of 12% (w/v). In some embodiments, trehalose is present at a concentration of 12.5% (w/v). In some embodiments, trehalose is present at a concentration of 13% (w/v). In some embodiments, trehalose is present at a concentration of 13.5% (w/v). In some embodiments, trehalose is present at a concentration of 14% (w/v). In some embodiments, trehalose is present at a concentration of 14.5% (w/v). In some embodiments, trehalose is present at a concentration of 15% (w/v).

### Polyols

In some embodiments, the stable formulation further comprises a polyol. Suitable polyol excipients include, for example, mannitol and sorbitol. In some embodiments, the stable formulation comprises a polyol at a concentration of 50 mM to 200 mM. In some embodiments, the stable formulation comprises a polyol at a concentration of 60 mM to 170 mM. In some embodiments, the polyol is present at a concentration of 2% (w/v) to 10% (w/v). In some embodiments, the polyol is present at a concentration of 3% (w/v) to 6% (w/v). In some embodiments, the polyol is present at a concentration of 2.5% (w/v), 3% (w/v), 3.5 (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), or 10% (w/v).

In some embodiments, the polyol comprises sorbitol. In some embodiments, the sorbitol is present at a concentration of 2% (w/v) to 10% (w/v). In some embodiments, the sorbitol is present at a concentration of 3% (w/v) to 6% (w/v). In some embodiments, the sorbitol is present at a concentration of 2.5% (w/v), 3% (w/v), 3.5 (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), or 10% (w/v). In some embodiments, sorbitol is present at a concentration of 2.5% (w/v). In some embodiments, sorbitol is present at a concentration of 3% (w/v). In some embodiments, sorbitol is present at a concentration of 3.5 (w/v). In some embodiments, sorbitol is present at a concentration of 4% (w/v). In some embodiments, sorbitol is present at a concentration of 4.5% (w/v). In some embodiments, sorbitol is present at a concentration of 5% (w/v). In some embodiments, sorbitol is present at a concentration of 5.5% (w/v). In some embodiments, sorbitol is present at a concentration of 6% (w/v). In some embodiments, sorbitol is present at a concentration of 6.5% (w/v). In some embodiments, sorbitol is present at a concentration of 7% (w/v). In some embodiments, sorbitol is present at a concentration of 7.5% (w/v). In some embodiments, sorbitol is present at a concentration of 8% (w/v). In some embodiments, sorbitol is present at a concentration of 8.5% (w/v). In some embodiments, sorbitol is present at a concentration of 9% (w/v). In some embodiments, sorbitol is present at a concentration of 9.5% (w/v). In some embodiments, sorbitol is present at a concentration of 10% (w/v).

In some embodiments, the polyol comprises mannitol. In some embodiments, the mannitol is present at a concentration of 2% (w/v) to 10% (w/v). In some embodiments, the mannitol is present at a concentration of 3% (w/v) to 6% (w/v). In some embodiments, the mannitol is present at a concentration of 2.5% (w/v), 3% (w/v), 3.5 (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), or 10% (w/v). In some embodiments, mannitol is present at a concentration of 2.5% (w/v). In some embodiments, mannitol is present at a concentration of 3% (w/v). In some embodiments, mannitol is present at a concentration of 3.5 (w/v). In some embodiments, mannitol is present at a concentration of 4% (w/v). In some embodiments, mannitol is present at a concentration of 4.5% (w/v). In some embodiments, mannitol is present at a concentration of 5% (w/v). In some embodiments, mannitol is present at a concentration of 5.5% (w/v). In some embodiments, mannitol is present at a concentration of 6% (w/v). In some embodiments, mannitol is present at a concentration of 6.5% (w/v). In some embodiments, mannitol is present at a concentration of 7% (w/v). In some embodiments, mannitol is present at a concentration of 7.5% (w/v). In some embodiments, mannitol is present at a concentration of 8% (w/v). In some embodiments, mannitol is present at a concentration of 8.5% (w/v). In some embodiments, mannitol is present at a concentration of 9% (w/v). In some embodiments, mannitol is present at a concentration of 9.5% (w/v). In some embodiments, mannitol is present at a concentration of 10% (w/v).

### Salts

In some embodiments, the stable formulation comprises less than 100 mM of a salt. In some embodiments, the stable formulation comprises less than 90 mM of a salt, less than 80 mM of a salt, less than 70 mM of a salt, less than 60 mM of a salt, less than 50 mM of a salt, less than 40 mM of a salt, less than 30 mM of a salt, less than 20 mM of a salt, less than 10 mM of a salt or less than 5 mM of a salt. In some embodiments, the formulation comprises only residual amounts of salts. In some embodiments, the formulation is substantially free of salt.

In other embodiments, the stable formulation comprises a salt. In some embodiments, the salt is present at a concentration of between 25 mM and 250 mM. In some embodiments, the salt is present at a concentration of between 35 mM and 100 mM. In some embodiments, salt is present at a concentration of 25 mM, 30 mM, 35 mM, 40 mM, 45, mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, 205 mM, 210 mM, 215 mM, 220 mM, 225 mM, 230 mM, 235 mM, 240 mM, 245 mM, or 250 mM.

In some embodiments, the salt comprises a halide. In some embodiments, the salt comprises NaCl. In some embodiments, NaCl is present at a concentration of between 25 mM and 250 mM. In some embodiments, NaCl is present at a concentration of between 35 mM and 100 mM. In some embodiments, NaCl is present at a concentration of 25 mM, 30 mM, 35 mM, 40 mM, 45, mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, 205 mM, 210 mM, 215 mM, 220 mM, 225 mM, 230 mM, 235 mM, 240 mM, 245 mM, or 250 mM. In some embodiments, NaCl is present at a concentration of 35 mM. In some embodiments, NaCl is present at a concentration of 100 mM.

### Amino Acids

In some embodiments, the stable formulation comprises one or more amino acids. In some embodiments, the one or more amino acids are present at a concentration between 10 mM and 250 mM. In some embodiments, the one or more amino acids are present at a concentration between 10 mM and 225 mM. In some embodiments, the one or more amino acids are present at a concentration between 10 mM and 200 mM. In some embodiments, the one or more amino acids are present at a concentration between 10 mM and 175 mM. In some embodiments, the one or more amino acids are present at a concentration between 10 mM and 150 mM. In some embodiments, the one or more amino acids are present at a concentration between 10 mM and 125 mM. In some embodiments, the one or more amino acids are present at a concentration between 10 mM and 100 mM. In some embodiments, the one or more amino acids are present at a concentration between 10 mM and 75 mM. In some embodiments, the one or more amino acids are present at a concentration between 10 mM and 50 mM. In some embodiments, the one or more amino acids are present at a concentration between 10 mM and 25 mM. In some embodiments, the one or more amino acids are present at a concentration between 20 mM and 250 mM. In some embodiments, the one or more amino acids are present at a concentration between 25 mM and 250 mM. In some embodiments, the one or more amino acids are present at a concentration between 50 mM and 250 mM. In some embodiments, the one or more amino acids are present at a concentration between 75 mM and 250 mM. In some embodiments, the one or more amino acids are present at a concentration between 100 mM and 250 mM. In some embodiments, the one or more amino acids are present at a concentration between 125 mM and 250 mM. In some embodiments, the one or more amino acids are present at a concentration between 150 mM and 250 mM. In some embodiments, the one or more amino acids are present at a concentration between 175 mM and 250 mM. In some embodiments, the one or more amino acids are present at a concentration between 200 mM and 250 mM. In some embodiments, the one or more amino acids are present at a concentration between 5 mM and 35 mM, between 10 mM and 35 mM, or between 15 mM and 30 mM.

In some embodiments, the stable formulation comprises one or more amino acids selected from arginine, glutamic acid, glycine, histidine, proline, and combinations thereof. In some embodiments, the stable formulation comprises arginine. In some embodiments, the stable formulation comprises glutamic acid. In some embodiments, the stable formulation comprises arginine and glutamic acid. In some embodiments, the stable formulation comprises histidine. In some embodiments, the stable formulation comprises glycine.

In some embodiments, the stable formulation comprising arginine comprises L-arginine. In some embodiments, the stable formulation comprising arginine comprises L-arginine hydrochloride. In some embodiments, the stable formulation comprises L-arginine. In some embodiments, the stable formulation comprises L-arginine hydrochloride. In some embodiments, the stable formulation comprises arginine at a concentration ranging from 10 mM to 250 mM. In some embodiments, the stable formulation comprises arginine at a concentration ranging from 10 mM to 225 mM. In some embodiments, the stable formulation comprises arginine at a concentration ranging from 10 mM to 200 mM. In some embodiments, the stable formulation comprises arginine at a concentration ranging from 10 mM to 175 mM. In some embodiments, the stable formulation comprises arginine at a concentration ranging from 10 mM to 150 mM. In some embodiments, the stable formulation comprises arginine at a concentration ranging from 10 mM to 125 mM. In some embodiments, the stable formulation comprises arginine at a concentration ranging from 10 mM to 100 mM. In some embodiments, the stable formulation comprises arginine at a concentration ranging from 10 mM to 75 mM. In some embodiments, the stable formulation comprises arginine at a concentration ranging from 10 mM to 50 mM. In some embodiments, the stable formulation comprises arginine at a concentration ranging from 10 mM to 25 mM. In some embodiments, the stable formulation comprises arginine at a concentration ranging from 10 mM to 20 mM. In some embodiments, the stable formulation comprises arginine at a concentration ranging from 20 mM to 250 mM. In some embodiments, the stable formulation comprises arginine at a concentration ranging from 25 mM to 250 mM. In some embodiments, the stable formulation comprises arginine at a concentration ranging from 50 mM to 250 mM. In some embodiments, the stable formulation comprises arginine at a concentration ranging from 75 mM to 250 mM. In some embodiments, the stable formulation comprises arginine at a concentration ranging from 100 mM to 250 mM. In some embodiments, the stable formulation comprises arginine at a concentration ranging from 125 mM to 250 mM. In some embodiments, the stable formulation comprises arginine at a concentration ranging from 150 mM to 250 mM. In some embodiments, the stable formulation comprises arginine at a concentration ranging from 175 mM to 250 mM. In some embodiments, the stable formulation comprises arginine at a concentration ranging from 200 mM to 250 mM. In some embodiments, the stable formulation comprises arginine at a concentration ranging from 225 mM to 250 mM. In some embodiments, the stable formulation comprises arginine at a concentration ranging from 20 mM to 30 mM, or 15 mM to 35 mM. In some embodiments, the stable formulation comprises arginine at a concentration of 25 mM. In some embodiments, the stable formulation comprises arginine at a concentration of 50 mM. In some embodiments, the stable formulation comprises arginine at a concentration of 75 mM. In some embodiments, the stable formulation comprises arginine at a concentration of 100 mM. In some embodiments, the stable formulation comprises arginine at a concentration of 125 mM. In some embodiments, the stable formulation comprises arginine at a concentration of 150 mM.

In some embodiments, the stable formulation comprises histidine. In some embodiments, the stable formulation comprises histidine at a concentration ranging from 5 mM to 25 mM. In some embodiments, the stable formulation comprises histidine at a concentration ranging from 5 mM to 20 mM. In some embodiments, the stable formulation comprises histidine at a concentration ranging from 5 mM to 15 mM. In some embodiments, the stable formulation comprises histidine at a concentration ranging from 5 mM to 10 mM. In some embodiments, the stable formulation comprises histidine at a concentration ranging from 10 mM to 25 mM. In some embodiments, the stable formulation comprises histidine at a concentration ranging from 15 mM to 25 mM. In some embodiments, the stable formulation comprises histidine at a concentration ranging from 20 mM to 25 mM. In some embodiments, the stable formulation comprises histidine at a concentration ranging from 15 mM to 25 mM, or between 10 mM to 30 mM. In some embodiments, the stable formulation comprises histidine at a concentration of 10 mM. In some embodiments, the stable formulation comprises histidine at a concentration of 20 mM.

### Antioxidants

In some embodiments, the stable formulation further comprises an antioxidant. In some embodiments, the antioxidant comprises ascorbic acid. In some embodiments, the antioxidant comprises L-methionine. In some embodiments, L-methionine is present at a concentration of 1 mM to 100 mM. In some embodiments, L-methionine is present at a concentration of 2 mM to 50 mM. In some embodiments, L-methionine is present at a concentration of 5 mM to 20 mM. In some embodiments, L-methionine is present at a concentration of, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM. In some embodiments, L-methionine is present at a concentration of 10 mM.

pH

In some embodiments, stable formulations were created with different pH levels in order to determine the effect of pH on formulation properties such as viscosity and stability (e.g., monomer purity, increase in high molecular weight (HMW) species, loss of main charge variant and charge distribution). In some embodiments, the stable formulation comprises a pH between 4.5 and 7.5. In some embodiments, the stable formulation comprises a pH between 5.0 and 7.5. In some embodiments, the stable formulation comprises a pH between 4.5 and 6.5. In some embodiments, the stable formulation comprises a pH between 5.0 and 6.0. In some embodiments, the stable formulation comprises a pH of 4.5. In some embodiments, the stable formulation comprises a pH of 5.0. In some embodiments, the stable formulation comprises a pH of 5.5. In some embodiments, the stable formulation comprises a pH of 6.0. In some embodiments, the stable formulation comprises a pH of 6.5. In some embodiments, the stable formulation comprises a pH of 7.0. In some embodiments, the stable formulation comprises a pH of 7.5.

### Osmolality

In some embodiments, the stable formulation comprises an osmolality ranging between 250 and 450 mOsmol/kg. In some embodiments, the stable formulation comprises an osmolality of between 275 and 410 mOsmol/kg. In some embodiments the stable formulation comprises an osmolality of 250, 260, 270, 280, 290, 300, 310, 320, 330, 340, 350, 360, 370, 380, 390, 400, 410, 420, 420, 440 or 450 mOsmol/kg. In some embodiments, the stable formulation comprises an osmolality of between 275 and 375 mOsmol/kg. In some embodiments, the stable formulation comprises an osmolality of between 310 and 410 mOsmol/kg.

### Viscosity

In some embodiments of the invention, the stable formulation is a liquid. In some embodiments of the invention, stable formulations were optimized in order to reduce viscosity, while maintaining high antibody concentration. Using a constant rate of 0.1 mL/minute, the force required to either aspirate (syringeability) or expel (injectability) the material from/into the syringe can be recorded. In some embodiments, lower viscosity allows for increased injectability of a formulation or effective sample transfer and preparation during manufacturing. In some embodiments, the stable formulation has a viscosity, as measured by microfluidic rheometer, ranging from 1 mPa*s to 70 mPa*s. In some embodiments, the stable formulation has a viscosity of less than 60 mPa*s, as measured by microfluidic rheometer. In some embodiments, the stable formulation has a viscosity of less than 50 mPa*s, as measured by microfluidic rheometer. In some embodiments, the stable formulation has a viscosity of less than 40 mPa*s, as measured by microfluidic rheometer. In some embodiments, the stable formulation has a viscosity of less than 30 mPa*s, as measured by microfluidic rheometer. In some embodiments, the stable formulation has a viscosity of less than 20 mPa*s, as measured by microfluidic rheometer. In some embodiments, the stable formulation has a viscosity of less than 15 mPa*s, as measured by microfluidic rheometer. In some embodiments, the stable formulation has a viscosity of less than 10 mPa*s, as measured by microfluidic rheometer. In some embodiments, the stable formulation has a viscosity of less than 5 mPa*s, as measured by microfluidic rheometer. In some embodiments, the stable formulation has a viscosity, as measured by a microfluidic rheometer, between approximately 1 and 30 mPa*s. In some embodiments, the stable formulation has a viscosity, as measured by a microfluidic rheometer, between approximately 2 and 30 mPa*s. In some embodiments, the stable formulation has a viscosity, as measured by a microfluidic rheometer, between approximately 4 and 30 mPa*s. In some embodiments, the stable formulation has a viscosity, as measured by a microfluidic rheometer, between approximately 6 and 30 mPa*s. In some embodiments, the stable formulation has a viscosity, as measured by a microfluidic rheometer, between approximately 8 and 30 mPa*s. In some embodiments, the stable formulation has a viscosity, as measured by a microfluidic rheometer, between approximately 10 and 30 mPa*s. In some embodiments, the stable formulation has a viscosity, as measured by a microfluidic rheometer, between approximately 12 and 30 mPa*s. In some embodiments, the stable formulation has a viscosity, as measured by a microfluidic rheometer, between approximately 14 and 30 mPa*s. In some embodiments, the stable formulation has a viscosity, as measured by a microfluidic rheometer, between approximately 16 and 30 mPa*s. In some embodiments, the stable formulation has a viscosity, as measured by a microfluidic rheometer, between approximately 18 and 30 mPa*s. In some embodiments, the stable formulation has a viscosity, as measured by a microfluidic rheometer, between approximately 20 and 30 mPa*s. In some embodiments, the stable formulation has a viscosity, as measured by a microfluidic rheometer, between approximately 22 and 30 mPa*s. In some embodiments, the stable formulation has a viscosity, as measured by a microfluidic rheometer, between approximately 24 and 30 mPa*s. In some embodiments, the stable formulation has a viscosity, as measured by a microfluidic rheometer, between approximately 26 and 30 mPa*s. In some embodiments, the stable formulation has a viscosity, as measured by a microfluidic rheometer, between approximately 28 and 30 mPa*s. In some embodiments, the stable formulation has a shear rate of less than 1000 s⁻¹ at 25° C.

In some embodiments, the stable formulation has the viscosity such that it is injectable via a regular syringe and needle combination, the needle having a nominal internal diameter of 0.1 to 0.6 mm. In some embodiments, the stable formulation is injectable by a needle having an internal diameter of 0.1, or 0.2, or 0.3, or 0.4 or 0.5, or 0.6 mm. In some embodiments, the stable formulation has a viscosity such that it is injectable via a regular syringe and needle combination, the needle having a nominal internal diameter of 0.18 to 0.3 mm. In some embodiments, the stable formulation has a viscosity such that it is injectable using a needle having an internal diameter between 0.184 mm to 0.260 mm diameter. In some embodiments, the stable formulation has the viscosity such that it is injectable via a regular syringe and needle combination, the needle having a nominal internal diameter of 0.184 to 0.210 mm. In some embodiments, the stable formulation has a viscosity such that it is injectable using 27G ½″ gauge needle and a 2 mL syringe. In some embodiments, the pound force required to inject the stable formulation at a constant rate of 0.1 mL/second is less than 8.0 pound force. In some embodiments, the pound force required to inject the stable formulation at a constant rate of 0.1 mL/second is less than 8 pound force, less than 7.5 pound force, or less than 6.9 pound force, less than 6.8 pound force, less than 6.7 pound force, less than 6.7 pound force, less than 6.6 pound force or less than 6.5 pound force.

### Stability

In some embodiments of the invention, the stable formulation was optimized in order to increase stability. The stability of an antibody formulation can be quantified in several ways. In some embodiments, stability of an antibody formulation is characterized by the amount of HMW species of an anti-TNFα antibody or the rate of increase of the amount of HMW species of an anti-TNFα antibody. For the purpose of the present application, the term high molecular weight species (HMW) of the product and “aggregates” are used interchangeably.

In certain embodiments, the rate of increase of HMW species is determined at 2, 4, 6 week, 2 months or 3 months in storage and at approximately 2-8° C., 25° C. or 40° C. In some embodiments, the stable formulation is stable following storage while either inverted or upright. In some embodiments, the stable formulation is stable following agitation at a speed of 100 rpm for up to 3 days. In some embodiments, the stable formulation is stable following 1, 2, 3, 4, or 5 freeze/thaw cycles.

In some embodiments, stability of an antibody formulation is characterized by charge distribution, e.g., a change in the amount of the charge variant peaks of the antibody. In some embodiments, stability of an antibody formulation is characterized by high accuracy liquid particle counter (HIAC), size exclusion ultra performance liquid chromatography (SE-UPLC), and/or imaged capillary isoelectric focusing (iCIEF). In some embodiments, stability of an antibody formulation is characterized by partial dissociation as measured by sodium-dodecyl sulfate capillary electrophoresis (CE-SDS) and/or sodium-dodecyl sulfate polyacrylamide gel electrophoresis (SDS-PAGE).

The stability of an anti-TNFα antibody, and the capability of the formulation to maintain stability of the anti-TNFα antibody, may be assessed over extended periods of time (e.g., weeks or months). In the context of a formulation, a stable formulation is one in which the antibody therein essentially retains its physical and/or chemical integrity and biological activity upon storage and during processes such as freeze/thaw, mechanical mixing and lyophilization. Antibody stability can be measured by formation of high molecular weight (HMW) aggregates, shift of charge profiles, and change in particle size.

Stability of an antibody may be assessed relative to the biological activity or physiochemical integrity of the antibody over extended periods of time. For example, stability at a given time point may be compared against stability at an earlier time point (e.g., upon formulation day 0), against unformulated antibody, or against a differently formulated antibody and the results of this comparison expressed as a percentage. Preferably, the antibody formulations of the present invention maintain at least 100%, at least 99%, at least 98%, at least 97% at least 95%, at least 90%, at least 85%, at least 80%, at least 75%, at least 70%, at least 65%, at least 60%, at least 55% or at least 50% of the antibody's biological activity, physiochemical integrity, and/or particle size over an extended period of time (e.g., as measured over at least 1, 2, 3, 4, 5, 6, 7, 8, 9, 10, 11, 12, 18, 24, or 36 months, at room temperature or under accelerated storage conditions). In some embodiments, the percent values indicating protein levels as disclosed herein or throughout the specification, are expressed relative to the total protein in the formulation. In some embodiments, the relative values of any particular species of the product, as disclosed herein or throughout the specification, such as the monomeric IgG form or species, or the high molecular weight (HMW) form, or the aggregated forms, are expressed in relation to the respective values of the total product. In some embodiments, the percent values of any particular species of the antibody are expressed relative to the total amount of all antibody-related species in the formulation.

In some embodiments, SEC-UPLC is used to measure the percentage of monomer or the percentage of HMW species of the anti-TNFα antibody of the formulation. In some embodiments, greater than 90%, equal to or greater than 91%, equal to or greater than 92%, equal to or greater than 93%, equal to or greater than 94%, equal to or greater than 95%, equal to or greater than 96%, equal to or greater than 97%, equal to or greater than 98%, equal to or greater than 99%, or equal to or greater than 99.5% of the anti-TNFα antibody exists as a monomer. In some embodiments, greater than or equal to 90% of the anti-TNFα antibody exists as a monomer. In some embodiments, less than 10%, 9%, 8%, 7%, 6%, 5%, 4%, 3%, 2%, 1%, or 0.5% of the anti-TNFα antibody exists as HMW species. In some embodiments, less than or equal to 5% of the anti-TNFα antibody exists as a HMW species. In some embodiments, at least 90%, 92%, 94%, 95%, 96%, 97%, 98%, or 99% of the anti-TNFα antibody exists as monomer in a stable formulation. In some embodiments, at least 90% of the anti-TNFα antibody exists as monomer in a stable formulation.

In some embodiments, the formulation is stable at 2-8° C. for more than 3 months. In some embodiments, the formulation is stable at 2-8° C. for more than 6 months. In some embodiments, the formulation is stable at 2-8° C. for more than 9 months. In some embodiments, the formulation is stable at 2-8° C. for more than 12 months. In some embodiments, the formulation is stable at 2-8° C. for more than 24 months. In some embodiments, the formulation is stable at 2-8° C. for more than 36 months. In some embodiments, the amount of HMW species in a formulation increases less than 10%, 9%, 8%, 7%, 6%, 5%, 4%, 3%, 2%, 1%, or 0.5% upon storage at 2-8° C. for more than 3 months. In some embodiments, the amount of HMW species in a formulation increases less than 10%, 9%, 8%, 7%, 6%, 5%, 4%, 3%, 2%, 1%, or 0.5% upon storage at 2-8° C. for more than 6 months. In some embodiments, the amount of HMW species in a formulation increases less than 10%, 9%, 8%, 7%, 6%, 5%, 4%, 3%, 2%, 1%, or 0.5% upon storage at 2-8° C. for more than 9 months. In some embodiments, the amount of HMW species in a formulation increases less than 10%, 9%, 8%, 7%, 6%, 5%, 4%, 3%, 2%, 1%, or 0.5% upon storage at 2-8° C. for more than 12 months. In some embodiments, the amount of HMW species in a formulation increases less than 10%, 9%, 8%, 7%, 6%, 5%, 4%, 3%, 2%, 1%, or 0.5% upon storage at 2-8° C. for more than 24 months. In some embodiments, the amount of HMW species in a formulation increases less than 10%, 9%, 8%, 7%, 6%, 5%, 4%, 3%, 2%, 1%, or 0.5% upon storage at 2-8° C. for more than 36 months.

In some embodiments, storage of the formulation at 2-8° C. for at least 3 months results in the formulation comprising less than 5% aggregates, and greater than 90% intact antibody. In some embodiments, storage of the formulation at 2-8° C. for at least 3 months results in the formulation comprising less than 3% aggregates, and greater than 97% intact antibody. In some embodiments, storage of the formulation at 2-8° C. for at least 3 months results in the formulation comprising less than 2% aggregates, and greater than 98% intact antibody.

In some embodiments, storage of the formulation at 2-8° C. for about at least 6 months results in the formulation comprising less than 5% aggregates, and greater than 90% intact antibody. In some embodiments, storage of the formulation at 2-8° C. for at least 6 months results in the formulation comprising less than 3% aggregates, and greater than 97% intact antibody. In some embodiments, storage of the formulation at 2-8° C. for at least 6 months results in the formulation comprising less than 2% aggregates, and greater than 98% intact antibody.

In some embodiments, storage of the formulation at 2-8° C. for at least 9 months results in the formulation comprising less than 5% aggregates, and greater than 90% intact antibody. In some embodiments, storage of the formulation at 2-8° C. for at least 9 months results in the formulation comprising less than 3% aggregates, and greater than 97% intact antibody. In some embodiments, storage of the formulation at 2-8° C. for at least 9 months results in the formulation comprising less than 2% aggregates, and greater than 98% intact antibody.

In some embodiments, storage of the formulation at 2-8° C. for at least 12 months results in the formulation comprising less than 5% aggregates, and greater than 90% intact antibody. In some embodiments, storage of the formulation at 2-8° C. for at least 12 months results in the formulation comprising less than 3% aggregates, and greater than 97% intact antibody. In some embodiments, storage of the formulation at 2-8° C. for at least 12 months results in the formulation comprising less than 2% aggregates, and greater than 98% intact antibody.

In some embodiments, storage of the formulation at 2-8° C. for at least 24 months results in the formulation comprising less than 5% aggregates, and greater than 90% intact antibody. In some embodiments, storage of the formulation at 2-8° C. for at least 24 months results in the formulation comprising less than 3% aggregates, and greater than 97% intact antibody. In some embodiments, storage of the formulation at 2-8° C. for at least 24 months results in the formulation comprising less than 2% aggregates, and greater than 98% intact antibody.

In some embodiments, storage of the formulation at 2-8° C. for at least 36 months results in the formulation comprising less than 5% aggregates, and greater than 90% intact antibody. In some embodiments, storage of the formulation at 2-8° C. for at least 36 months results in the formulation comprising less than 3% aggregates, and greater than 97% intact antibody. In some embodiments, storage of the formulation at 2-8° C. for at least 36 months results in the formulation comprising less than 2% aggregates, and greater than 98% intact antibody.

In some embodiments, the formulation is stable at 25° C. for more than 3 months. In some embodiments, the amount of HMW species in a formulation increases less than 10%, 9%, 8%, 7%, 6%, 5%, 4%, 3%, 2%, 1%, or 0.5% upon storage at 25° C. for more than 3 months.

In some embodiments, storage of the formulation at 25° C. for at least 3 months results in the formulation comprising less than 5% aggregates, and greater than 90% intact antibody. In some embodiments, storage of the formulation at 25° C. for at least 3 months results in the formulation comprising less than 3% aggregates, and greater than 97% intact antibody. In some embodiments, storage of the formulation at 25° C. for at least 3 months results in the formulation comprising less than 2% aggregates, and greater than 98% intact antibody.

In some embodiments, the formulation is stable at 40° C. for more than 4 weeks. In some embodiments, upon storage at 40° C. for 4 weeks, the amount of HMW species in the formulation increases approximately between 0.3% to 0.7%, approximately between 0.3% to 0.6%, or approximately between 0.3% to 0.5%. In some embodiments, storage of the formulation at 40° C. for at least 4 weeks results in the formulation comprising less than 5% aggregates, and greater than 90% intact antibody. In some embodiments, storage of the formulation at 40° C. for at least 4 weeks results in the formulation comprising less than 4% aggregates, and greater than 96% intact antibody.

In some embodiments, reduced CE-SDS is used to measure the percentage of the anti-TNFα antibody of the formulation that comprise a light chain and a heavy chain, or the total percentage of minor species. In some embodiments, the percentage of anti-TNFα antibody comprising a light chain and a heavy chain is greater than or equal to 90%. In some embodiments, the total percentage of minor species is less than or equal to 10%.

In some embodiments, non-reduced CE-SDS is used to measure the purity of the main peak, or the total percentage of LMW species. In some embodiments, the main peak percentage is greater than or equal to 90%. In some embodiments, total LMW species is less than or equal to 10%.

In some embodiments, the formulation is essentially free of all visible particles.

In some embodiments, cation exchange chromatography is used to measure the charge variants of the of the anti-TNFα antibody of the formulation. In some embodiments, the main peak percentage of the of the anti-TNFα antibody is greater than or equal to 40%. In some embodiments, the acidic peak percentage of the of the anti-TNFα antibody is less than or equal to 50%. In some embodiments, the basic peak percentage of the of the anti-TNFα antibody is less than or equal to 20%.

### Dose

In some embodiments, the anti-TNFα antibody is administered at a dose of about 10 mg to about 300 mg. In some embodiments, the anti-TNFα antibody is administered at a dose of 20 mg to 240 mg. In some embodiments, the anti-TNFα antibody is administered of 20 mg to 180 mg. In some embodiments, the anti-TNFα antibody is administered at a dose of 30 mg to 60 mg. In some embodiments, the anti-TNFα antibody is administered at a dose of 50 mg to 100 mg. In some embodiments, the anti-TNFα antibody is administered at a dose of 90 mg to 150 mg. In some embodiments, the anti-TNFα antibody is administered at a dose of 130 mg to 190 mg. In some embodiments, the anti-TNFα antibody is administered at a dose of 180 mg to 220 mg. In some embodiments, the anti-TNFα antibody is administered at a dose of 200 mg to 240 mg.

An exemplary treatment regime entails administration once per week, once every two weeks, once every three weeks, once every four weeks, once a month, once every four weeks, once every five weeks, once every six weeks, once every seven weeks, once every eight weeks, or once every two months. In some embodiments, treatment regimen entails administration once every other week, once every three weeks, once every four weeks, once every month, once every five weeks, once every six weeks, once every seven weeks, once every eight weeks, or once every other month.

In some embodiments the anti-TNFα antibody is administered no more frequently than once every other week. In some embodiments the anti-TNFα antibody is administered no more frequently than once every three weeks. In some embodiments the anti-TNFα antibody is administered no more frequently than once every four weeks. In some embodiments the anti-TNFα antibody is administered no more frequently than once every month. In some embodiments the anti-TNFα antibody is administered no more frequently than once every five weeks. In some embodiments the anti-TNFα antibody is administered no more frequently than once every six weeks. In some embodiments the anti-TNFα antibody is administered no more frequently than once every seven weeks. In some embodiments the anti-TNFα antibody is administered no more frequently than once every eight weeks. In some embodiments the anti-TNFα antibody is administered no more frequently than once every other month.

In some embodiments the anti-TNFα antibody is administered at a dose of 40 mg, 80 mg, or 120 mg every four weeks (Q4W). In some embodiments the anti-TNFα antibody is administered at a dose of 40 mg every four weeks (Q4W). In some embodiments the anti-TNFα antibody is administered at a dose of 80 mg every four weeks (Q4W). In some embodiments the anti-TNFα antibody is administered at a dose of 120 mg every four weeks (Q4W). In some embodiments the anti-TNFα antibody is administered every four weeks for three administrations (Q4W*3).

In some embodiments, methods described herein comprise administration of a loading dose of an anti-TNFα antibody.

### Methods of Administration

In some embodiments, anti-TNFα antibody 1 is administered subcutaneously. In some embodiments, a composition of the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include subcutaneous, intravenous, intramuscular, intradermal, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, I R. Robinson, ed., Marcel Dekker, Inc., New York, 1978.

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al, (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); ρ120 (Schreier et al. (1994) J. Biol. Chem. 269:9090); see also K. Keinanen; M. L. Laukkanen (1994) FEBS Lett. 346:123; J J. Killion; L J. Fidler (1994 J Immunomethods 4:273.

### Autoimmune Diseases

Inappropriate or excessive activation of TNF-α signaling is associated with chronic inflammation and can eventually lead to the development of pathological complications such as autoimmune diseases. Due to the involvement of TNF-α in the pathogenesis of autoimmune diseases, TNF-α inhibitors have been successfully developed and applied in the clinical treatment of autoimmune diseases such as Crohn's disease (CD) and RA. Provided herein are methods of treating autoimmune diseases comprising administering anti-TNFα antibody 1. In desirable embodiments, the mammal has an autoimmune disease or an increased risk for an autoimmune disease.

Exemplary autoimmune diseases include Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barré, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, Wegener's Granulomatosis, and myasthenia gravis. In some embodiments, any metabolic disorder that is due to the injury or damage of the cells of a predetermined type or due to the autoimmune disease is controlled. In some embodiments, the mammal has an established autoimmune disease (e.g., the mammal has symptoms of the autoimmune disease). In some embodiments, the mammal does not have an established autoimmune disease. In particular embodiments, the mammal does not have cancer or AIDS. Desirably, the mammal is a human. In some embodiments, the autoimmune disease is selected from hidradenitis suppurativa, rheumatoid arthritis (RA), psoriatic arthritis (PsA). In some embodiments, the autoimmune disease is ankylosing spondylitis (AS). In some embodiments, the autoimmune disease is Crohn's disease. In some embodiments, the autoimmune disease is psoriasis. In some embodiments, the autoimmune disease is selected from hidradenitis suppurativa (HS), rheumatoid arthritis (RA), juvenile idiopathic arthritis, psoriatic arthritis (PsA), ankylosing spondylitis (AS), refractory asthma, Crohn's disease, psoriasis, ulcerative colitis, and uveitis. In some embodiments, the autoimmune disease is rheumatoid arthritis (RA).

### EXAMPLES

Examples are provided below to illustrate the present invention. These examples are not meant to constrain the present invention to any particular application or theory of operation.

### Example 1: Formulation Develop of Anti-TNFα Antibody 1

This example details the development of a high-concentration formulation for anti-TNFα Antibody 1 suitable for subcutaneous administration. The final formulation for anti-TNFα Antibody 1 was derived from conducting a solubility study followed by a pH/buffer screening study and then an excipient/surfactant strength screening study.

### Solubility Study

An initial solubility study was performed to gain a preliminary understanding of the protein solubility as well as assess the feasibility of high concentration formulation develop in view of potential challenges (i.e., viscosity, stability). Anti-TNFα antibody 1 was prepared at four protein concentrations—50, 100, 150 and 200 mg/mL, corresponding to F1-1, F1-2, F1-3, and F1-4, respectively—and the required amount of surfactant stock solution (5% (w/w) PS80) was calculated, added and well-mixed. Each of the prepared samples was filtered with a 0.22 μm polyvinylidene fluoride (PVDF) filter and filled into 2 R glass vials (1 mL per vial), then stoppered, capped and labeled immediately. Viscosity and osmolality, appearance, and protein concentration, and SE-HPLC were assessed.

TABLE 2
Viscosity and osmolality.

| | | |
|---|---|---|
| | Viscosity | Osmolality |
| Formulation No. | (cP, 25° C.) | (mOsm/Kg) |

| | |
|---|---|
| (Protein conc., mg/mL) | T0 |

| | | |
|---|---|---|
| F1-1 (50) | 1.77 | 277 |
| F1-2 (100) | 3.56 | 307 |
| F1-3 (150) | 10.36 | 335 |
| F1-4 (200) | 51.25 | 392 |

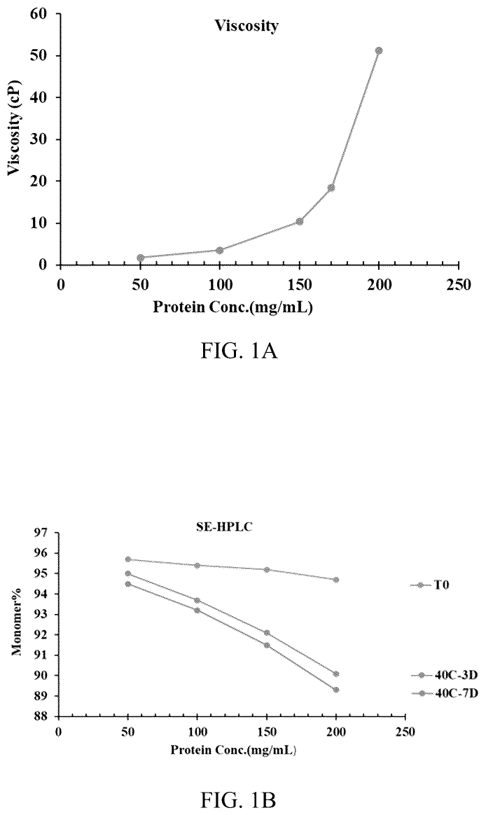

The osmolality increased gradually with the increase of protein concentration and the highest osmolality was 392 mOsm/kg which was still acceptable for the subcutaneous injection. The viscosity increased rapidly with the increase of protein concentration as shown in FIG. 1A.

TABLE 3
Appearance and Protein Concentration.

| | | | | Protein conc. | | |
|---|---|---|---|---|---|---|
| | Appearance | | | (mg/mL) | | |
| | | 40° C. | | | 40° C. | |
| Formulation No. | T0 | 3 D | 7 D | T0 | 3 D | 7 D |
| F1-1 | SY, SO, FP | SY, SO, FP | SY, SO, FP | 50.0 | 50.6 | 51.0 |
| F1-2 | SY, SO, FP | SY, SO, FP | SY, SO, FP | 98.3 | 101.5 | 101.7 |
| F1-3 | SY, SO, FP | SY, SO, FP | SY, SO, FP | 148.6 | 153.4 | 162.5 |
| F1-4 | Y, SO, FP | Y, SO, FP | Y, SO, FP | 208.4 | 210.0 | 209.8 |

Abbreviations: Y = yellow, SY = slightly yellow, SO = slightly opalescent, FP = free of visible particles.

All samples were slightly opalescent, free of visible particles and the color changed from slight yellow to yellow with the increase of protein concentration. No obvious change was found in protein concentration for all samples after incubation at 40° C. for 7 days.

TABLE 4
SE-HPLC.

| | SE-HPLC (Monomer %/HMW %/LMW %) | | |
|---|---|---|---|
| Formulation | | 40° C. | |
| No. | T0 | 3 D | 7 D |
| F1-1 | 95.7/4.3/0.0 | 95.0 (↓0.7)/4.9/0.2 | 94.5 (↓1.2)/5.2/0.3 |
| F1-2 | 95.4/4.6/0.0 | 93.7 (↓1.7)/6.2/0.2 | 93.2 (↓2.2)/6.5/0.3 |
| F1-3 | 95.2/4.7/0.0 | 92.1 (↓3.1)/7.7/0.2 | 91.5 (↓3.7)/8.2/0.3 |
| F1-4 | 94.7/5.3/0.0 | 90.1 (↓4.6)/9.7/0.2 | 89.3 (↓5.4)/10.4/0.3 |

FIG. 1B shows stability data for the percent monomer at T0, day 3, and day 7 when stored at 40° C. Based on the solubility study data, anti-TNFα antibody 1 could be concentrated to 200 mg/mL.

pH/Buffer Screening Study

Different pH/buffer systems were screened under stress conditions (40° C. incubation). A protein concentration 50 mg/mL anti-TNFα Antibody 1 was used in this study for the purposes of conserving available protein.

Anti-TNFα Antibody 1 was firstly buffer-exchanged into 12 buffers including 20 mM succinate buffer (pH 5.0, pH 5.5, pH 6.0), 20 mM acetate buffer (pH 4.5, pH 5.0, pH 5.5), 20 mM histidine buffer (pH 5.5, pH 6.0, pH 6.5), and 20 mM PB (pH 6.5, pH 7.0, pH 7.5), and then adjusted to target protein conc. (50 mg/mL), respectively (as indicated in Table 5). Each of the prepared samples was filtered with a 0.22 μm PVDF filter and filled into 2 R glass vials (1 mL per vial), then stoppered, capped and labeled immediately.

TABLE 5
Formulation candidates assessed in pH/buffer screening study.

| Formulation No. | Buffer system | Target pH |
|---|---|---|
| F2-1 | 20 mM Succinate | 5.0 |
| F2-2 | | 5.5 |
| F2-3 | | 6.0 |
| F2-4 | 20 mM Acetate | 4.5 |
| F2-5 | | 5.0 |
| F2-6 | | 5.5 |
| F2-7 | 20 mM Histidine | 5.5 |
| F2-8 | | 6.0 |
| F2-9 | | 6.5 |
| F2-10 | 20 mM Phosphate Buffer (PB) | 6.5 |
| F2-11 | | 7.0 |
| F2-12 | | 7.5 |

Samples were stored at 40° C. for up to 4 weeks. Samples were pulled at each time point and kept at −70° C. before analysis. Samples were tested for appearance, pH, protein concentration, SE-HPLC, iCIEF, caliper-SDS-NR&R, and DSC.

All samples remained slightly yellow, slightly opalescent and free of visible particles after being stored at 40° C. for 4 weeks.

In addition, the formulations (F2-1-F2-12) were assessed for the pH and protein concentration (Table 6), and tested using SE-HPLC (Table 7), iCIEF (Table 8), caliper-SDS-NR (Table 9) and DSC (Table 10) as shown below.

TABLE 6
pH and protein concentration.

| | pH | | | | Protein conc. (mg/mL) | | | |
|---|---|---|---|---|---|---|---|---|
| | | 40° C. | | | | 40° C. | | |
| Formulation No. | T0 | 1 W | 2 W | 4 W | T0 | 1 W | 2 W | 4 W |
| F2-1 | 5.1 | 5.0 | 5.0 | 5.0 | 49.6 | 50.0 | 49.6 | 47.0 |
| F2-2 | 5.6 | 5.6 | 5.6 | 5.5 | 50.1 | 50.3 | 50.2 | 47.9 |
| F2-3 | 6.0 | 6.0 | 6.0 | 5.9 | 49.7 | 50.1 | 50.0 | 47.7 |
| F2-4 | 4.6 | 4.6 | 4.6 | 4.6 | 49.9 | 50.5 | 50.0 | 47.3 |
| F2-5 | 5.1 | 5.1 | 5.1 | 5.1 | 49.3 | 49.6 | 49.7 | 47.0 |
| F2-6 | 5.5 | 5.5 | 5.5 | 5.5 | 49.4 | 50.1 | 49.5 | 47.4 |
| F2-7 | 5.5 | 5.5 | 5.6 | 5.5 | 49.8 | 50.2 | 50.3 | 47.8 |
| F2-8 | 6.1 | 6.1 | 6.1 | 6.0 | 50.2 | 50.6 | 50.5 | 47.9 |
| F2-9 | 6.5 | 6.5 | 6.5 | 6.4 | 49.7 | 50.4 | 50.3 | 47.9 |
| F2-10 | 6.5 | 6.5 | 6.5 | 6.5 | 50.0 | 50.7 | 50.3 | 47.7 |
| F2-11 | 6.9 | 7.0 | 6.9 | 6.9 | 50.3 | 50.4 | 50.3 | 47.8 |
| F2-12 | 7.5 | 7.5 | 7.5 | 7.4 | 50.5 | 50.9 | 50.9 | 48.3 |

TABLE 7
SE-HPLC.

| | SE-HPLC (Monomer %/HMW %/LMW %) | | | |
|---|---|---|---|---|
| Formulation | | 40° C. | | |
| No. | T0 | 1 W | 2 W | 4 W |
| F2-1 | 95.7/4.2/0.0 | 94.9(↓0.8)/4.8/0.3 | 94.5(↓1.2)/5.0/0.5 | 90.5(↓5.2)/5.8/3.8 |
| F2-2 | 95.7/4.3/0.0 | 94.2(↓1.5)/5.5/0.2 | 93.8(↓1.9)/5.8/0.4 | 90.3(↓5.4)/6.3/3.4 |
| F2-3 | 95.3/4.6/0.0 | 93.5(↓1.8)/6.2/0.2 | 93.2(↓2.1)/6.5/0.4 | 89.4(↓5.9)/7.1/3.5 |
| F2-4 | 95.9/4.1/0.0 | 95.5(↓0.4)/4.2/0.3 | 95.1(↓0.8)/4.3/0.6 | 90.8(↓5.1)/4.9/4.4 |
| F2-5 | 95.7/4.2/0.0 | 95.4(↓0.3)/4.4/0.2 | 95.2(↓0.5)/4.4/0.4 | 91.7(↓4.0)/4.7/3.6 |
| F2-6 | 95.6/4.4/0.0 | 94.8(↓0.8)/4.9/0.2 | 94.5(↓1.1)/5.1/0.4 | 91.2(↓4.4)/5.5/3.4 |
| F2-7 | 95.8/4.2/0.0 | 95.5(↓0.3)/4.3/0.2 | 95.2(↓0.6)/4.3/0.4 | 91.9(↓3.9)/4.6/3.5 |
| F2-8 | 95.8/4.2/0.0 | 95.2(↓0.6)/4.5/0.3 | 94.8(↓1.0)/4.7/0.5 | 91.2(↓4.6)/5.0/3.8 |
| F2-9 | 95.7/4.3/0.0 | 94.8(↓0.9)/4.9/0.3 | 94.3(↓1.4)/5.1/0.6 | 89.5(↓6.2)/5.5/5.0 |
| F2-10 | 95.2/4.8/0.0 | 92.8(↓2.4)/7.0/0.2 | 92.4(↓2.8)/7.3/0.3 | 89.0(↓6.2)/8.0/3.0 |
| F2-11 | 95.3/4.6/0.0 | 92.3(↓3.0)/7.4/0.2 | 91.8(↓3.5)/7.8/0.4 | 87.9(↓7.4)/8.7/3.3 |
| F2-12 | 95.3/4.6/0.0 | 92.1(↓3.2)/7.6/0.3 | 91.3(↓4.0)/8.2/0.5 | 86.7(↓8.6)/9.3/4.0 |

TABLE 8
iCIEF.

| | iCIEF (Main peak %/AP %/BP %) | | | |
|---|---|---|---|---|
| Formulation | | 40° C. | | |
| No. | T0 | 1 W | 2 W | 4 W |
| F2-1 | 58.1/32.1/9.8 | 57.3(↓0.8)/34.7/8.0 | 45.6(↓12.5)/39.3/15.2 | 32.8(↓25.3)/52.4/14.8 |
| F2-2 | 59.0/33.6/7.4 | 55.1(↓3.9)/35.1/9.8 | 50.0(↓9.0)/38.3/11.7 | 37.9(↓21.1)/49.2/13.0 |
| F2-3 | 61.9/29.5/8.5 | 55.3(↓6.6)/34.6/10.1 | 48.8(↓13.1)/38.2/13.0 | 37.1(↓24.8)/46.9/16.0 |
| F2-4 | 56.7/35.4/7.9 | 55.0(↓1.7)/34.0/11.0 | 49.0(↓7.7)/36.5/14.5 | 37.7(↓19.0)/42.9/19.3 |
| F2-5 | 58.8/32.7/8.5 | 55.5(↓3.3)/33.7/10.8 | 49.6(↓9.2)/36.3/14.1 | 40.0(↓18.8)/42.1/18.0 |
| F2-6 | 58.1/34.1/7.8 | 56.2(↓1.9)/33.8/10.0 | 54.0(↓4.1)/37.5/8.5 | 41.2(↓16.9)/44.4/14.4 |
| F2-7 | 58.5/31.5/10.0 | 55.2(↓3.3)/34.5/10.3 | 54.5(↓4.0)/35.8/9.7 | 39.4(↓19.1)/45.3/15.2 |
| F2-8 | 57.7/35.5/6.8 | 54.4(↓3.3)/36.7/8.9 | 53.0(↓4.7)/38.5/8.4 | 40.2(↓17.5)/48.8/11.0 |
| F2-9 | 57.9/31.5/10.5 | 53.4(↓4.5)/37.1/9.5 | 48.2(↓9.7)/40.4/11.4 | 35.1(↓22.8)/50.1/14.8 |
| F2-10 | 58.0/33.7/8.2 | 51.9(↓6.1)/38.4/9.6 | 45.9(↓12.1)/42.8/11.3 | 33.9(↓24.1)/53.5/12.5 |
| F2-11 | 58.0/33.7/8.2 | 47.5(↓10.5)/41.7/10.8 | 40.6(↓17.4)/48.4/11.0 | 26.7(↓31.3)/58.5/14.7 |
| F2-12 | 58.8/32.9/8.3 | 41.8(↓17.0)/47.7/10.5 | 32.2(↓26.6)/25.2/42.6 | 25.2(↓33.6)/44.6/30.2 |

TABLE 9
caliper-SDS (NR&R).

| | Caliper-SDS-NR (purity %) | | | | Caliper-SDS-R (LC + HC)% | | | |
|---|---|---|---|---|---|---|---|---|
| Formulation No. | | 40° C. | | | | 40° C. | | |
| (Buffer/pH) | T0 | 1 W | 2 W | 4 W | T0 | 1 W | 2 W | 4 W |
| F2-1 | 98.1 | 95.8 | 95.0 | 93.4 | 99.4 | 99.3 | 99.0 | 98.4 |
| F2-2 | 97.9 | 96.5 | 95.2 | 93.3 | 99.3 | 99.4 | 99.2 | 98.7 |
| F2-3 | 97.9 | 96.5 | 95.4 | 92.0 | 99.3 | 98.8 | 99.1 | 98.4 |
| F2-4 | 98.0 | 96.1 | 96.0 | 92.7 | 99.5 | 99.3 | 99.3 | 98.7 |
| F2-5 | 97.8 | 96.5 | 95.9 | 93.7 | 99.2 | 99.3 | 99.3 | 98.7 |
| F2-6 | 98.0 | 96.4 | 95.8 | 92.8 | 99.5 | 99.2 | 99.1 | 98.1 |
| F2-7 | 98.1 | 96.9 | 96.0 | 93.5 | 99.5 | 99.5 | 99.4 | 98.8 |
| F2-8 | 98.0 | 95.8 | 95.9 | 92.2 | 99.5 | 99.0 | 99.2 | 97.4 |
| F2-9 | 98.0 | 96.0 | 95.6 | 91.1 | 98.0 | 98.8 | 99.2 | 97.6 |
| F2-10 | 98.0 | 96.9 | 94.5 | 92.4 | 98.6 | 99.3 | 99.0 | 96.1 |
| F2-11 | 98.0 | 96.3 | 94.3 | 89.7 | 98.9 | 98.7 | 98.6 | 94.1 |
| F2-12 | 98.0 | 96.2 | 93.4 | 85.6 | 99.1 | 99.3 | 98.2 | 93.9 |

TABLE 10
DSC.

| Formulation No. | T_{Onset} | Tₘ1 | Tₘ2 |
|---|---|---|---|
| (Buffer/pH) | (° C.) | (° C.) | (° C.) |
| F2-1 | 56.6 | 66.8 | 72.9 |
| F2-2 | 61.2 | 73.2 | 78.9 |
| F2-3 | 63.4 | 73.7 | 78.9 |
| F2-4 | 56.5 | 65.8 | 73.4 |
| F2-5 | 60.4 | 73.8 | 79.9 |
| F2-6 | 63.3 | 74.2 | 79.8 |
| F2-7 | 58.4 | 67.1 | 73.3 |
| F2-8 | 61.9 | 74.1 | 80.5 |
| F2-9 | 63.1 | 74.8 | 80.7 |
| F2-10 | 65.2 | 74.3 | 78.7 |
| F2-11 | 65.4 | 74.3 | 78.5 |
| F2-12 | 60.6 | 73.4 | 77.9 |

### Excipients and Surfactant Strength Screening Study

Differing combinations of excipients and surfactants of various concentrations were screened to determine an optimal formulation to stabilize anti-TNFα Antibody 1. Different conditions including thermal stress (40° C. incubation), multiple freeze and thaw cycles, agitation and oxidation (H₂O₂ spiking) were used to assess stability. 170 mg/mL was set as the target protein concentration in this study. Formulation candidates F3-1-F3-12 were prepared as listed in Table 11.

TABLE 11
Formulation candidates.

| Formulation | Buffer system, pH, | | Surfactant |
|---|---|---|---|
| No. | protein conc. | Excipients | (w/v) |
| F3-1 | 20 mM histidine | 7% (w/v) sucrose | 0.02% PS80 |
| F3-2 | buffer, pH 5.5, | 7% (w/v) sucrose, 10 mM methionine | |
| F3-3 | 170 mg/mL | 7.7% (w/v) trehalose•2H₂O, 10 mM | |
| | | methionine | |
| F3-4 | | 4.5% (w/v) mannitol, 10 mM methionine | |
| F3-5 | | 4.5% (w/v) sorbitol, 10 mM methionine | |
| F3-6 | | 100 mM NaCl, 10 mM methionine | |
| F3-7 | | 100 mM arginine-HCl, 10 mM | |
| | | methionine | |
| F3-8 | | 4% (w/v) sucrose, 35 mM arginine-HCl, | |
| | | 10 mM methionine | |
| F3-9 | | 4% (w/v) sucrose, 35 mM NaCl, 10 mM | |
| | | methionine | |
| F3-10 | | 7% (w/v) sucrose | 0.01% PS80 |
| F3-11 | | | 0.04% PS80 |
| F3-12 | | | 0.06% PS80 |

For freeze/thaw (FT), samples were completely frozen at −40° C. and thawed at room temperature (RT) for up to 5 cycles (CYS). For thermal stress, samples were stored at 40° C. for up to 4 weeks. For agitation stress, samples were agitated at 300 rpm for up to 3 days. For H₂O₂ spiking study, samples (only for F3-1 and F3-2) were incubated with 5 ppm H₂O₂ for up to 4 weeks or 0.1% (w/v) H₂O₂ for 24 h at 25° C., respectively. Testing items including appearance, pH, protein conc., osmolality, viscosity, SE-HPLC, caliper-SDS-NR&R, iCIEF, liquid particle counting and DSC.

Following stress conditions, all formulations remained yellow, slightly opalescent and free of visible particles. Stronger opalescence was observed in F3-6-F3-9 and the formulation candidates were ranked as F3-6 (100 mM NaCl)>F3-7 (100 mM arginine-HCl)>F3-9 (35 mM NaCl)>F3-8 (35 mM arginine-HCl).

TABLE 12
pH and viscosity.

| | pH | | | | | | | | | | | |
|---|---|---|---|---|---|---|---|---|---|---|---|---|
| | | | | FT | | | | 0.1% (w/v) | | 5 ppm | | Viscosity |
| | | 40° C. | | 3 | 5 | Agitation | | H₂O₂ | | H₂O₂ | | (25° C., cP) |
| Formulation No. | T0 | 2 W | 4 W | CYS | CYS | 1 D | 3 D | 4 h | 24 h | 2 W | 4 W | T0 |
| F3-1 | 5.6 | 5.5 | 5.6 | 5.6 | 5.6 | 5.6 | 5.6 | 5.6 | 5.5 | 5.5 | 5.6 | 15.15 |
| F3-2 | 5.6 | 5.5 | 5.6 | 5.6 | 5.6 | 5.6 | 5.6 | 5.5 | 5.5 | 5.5 | 5.6 | 14.62 |
| F3-3 | 5.6 | 5.5 | 5.6 | 5.6 | 5.6 | 5.6 | 5.6 | / | / | / | / | 15.66 |
| F3-4 | 5.6 | 5.5 | 5.6 | 5.6 | 5.5 | 5.6 | 5.6 | / | / | / | / | 10.57 |
| F3-5 | 5.6 | 5.6 | 5.6 | 5.6 | 5.5 | 5.6 | 5.6 | / | / | / | / | 10.34 |
| F3-6 | 5.6 | 5.6 | 5.6 | 5.6 | 5.5 | 5.6 | 5.6 | / | / | / | / | 12.99 |
| F3-7 | 5.6 | 5.6 | 5.6 | 5.6 | 5.5 | 5.6 | 5.6 | / | / | / | / | 10.98 |
| F3-8 | 5.5 | 5.5 | 5.5 | 5.5 | 5.5 | 5.6 | 5.5 | / | / | / | / | 13.04 |
| F3-9 | 5.6 | 5.5 | 5.5 | 5.6 | 5.5 | 5.6 | 5.6 | / | / | / | / | 14.08 |
| F3-10 | 5.5 | 5.5 | 5.5 | 5.5 | 5.5 | 5.5 | 5.5 | / | / | / | / | 14.85 |
| F3-11 | 5.5 | 5.4 | 5.5 | 5.5 | 5.5 | 5.5 | 5.5 | / | / | / | / | 14.72 |
| F3-12 | 5.5 | 5.4 | 5.5 | 5.5 | 5.5 | 5.5 | 5.5 | / | / | / | / | 14.10 |

Anti-TNFα Antibody 1 protein concentration (approximately 170.0 mg/mL) remained relatively stable. The osmolality of these formulations were 287˜420 mOsm/kg, which were all within acceptable range for subcutaneous injection.

TABLE 13
Protein concentration and osmolality.

| | Protein conc. (mg/mL) | | | | | | | | | | | |
|---|---|---|---|---|---|---|---|---|---|---|---|---|
| | | | | FT | | | | 0.1% (w/v) | | 5 ppm | | |
| Formulation | | 40° C. | | 3 | 5 | Agitation | | H₂O₂ | | H₂O₂ | | mOsm/Kg |
| No. | T0 | 2 W | 4 W | CYS | CYS | 1 D | 3 D | 4 H | 24 H | 2 W | 4 W | T0 |
| F3-1 | 175.4 | 176.7 | 173.2 | 177.7 | 172.9 | 178.4 | 174.9 | 171.5 | 172.1 | 173.3 | 174.6 | 395 |
| F3-2 | 172.8 | 175.4 | 169.1 | 177.6 | 171.1 | 178.7 | 176.5 | 169.0 | 171.4 | 170.3 | 171.6 | 403 |
| F3-3 | 173.9 | 169.9 | 174.8 | 177.3 | 172.7 | 178.4 | 178.1 | / | / | / | / | 420 |
| F3-4 | 166.4 | 170.0 | 166.5 | 170.0 | 166.1 | 171.5 | 166.4 | / | / | / | / | 415 |
| F3-5 | 164.4 | 164.7 | 161.1 | 168.5 | 160.3 | 163.1 | 162.4 | / | / | / | / | 401 |
| F3-6 | 167.2 | 164.4 | 168.6 | 172.5 | 165.3 | 170.3 | 174.2 | / | / | / | / | 302 |
| F3-7 | 167.9 | 169.5 | 167.0 | 174.3 | 167.1 | 170.4 | 170.8 | / | / | / | / | 287 |
| F3-8 | 174.2 | 174.1 | 168.0 | 176.5 | 168.3 | 175.2 | 174.4 | / | / | / | / | 332 |
| F3-9 | 172.3 | 171.9 | 170.1 | 175.8 | 168.9 | 174.0 | 171.0 | / | / | / | / | 349 |
| F3-10 | 171.5 | 171.0 | 170.1 | 173.7 | 166.2 | 174.9 | 171.3 | / | / | / | / | 387 |
| F3-11 | 174.7 | 170.4 | 169.0 | 173.7 | 168.0 | 173.5 | 171.7 | / | / | / | / | 392 |
| F3-12 | 174.2 | 173.6 | 166.5 | 175.9 | 166.3 | 176.6 | 171.4 | / | / | / | / | 400 |

TABLE 14
Sub-visible particles.

| | Sub-visible particles counts (#/mL, ≥2 μm/≥10 μm/≥25 μm) | | | | | |
|---|---|---|---|---|---|---|
| | | | | | 0.1% (w/v) | 5 ppm |
| | | 40° C. | FT | Agitation | H₂O₂ | H₂O₂ |
| Formulation No. | T0 | 4 W | 5 CYS | 3 D | 24 h | 4 W |
| F3-1 | 183/12/1 | 80/1/0 | 104/6/0 | 53/4/1 | 751/72/1 | 360/63/1 |
| F3-2 | 14/1/1 | 63/0/0 | 120/2/0 | 98/1/0 | 221/13/0 | 105/6/2 |
| F3-3 | 26/0/0 | 59/1/0 | 40/3/0 | 26/1/0 | / | / |
| F3-4 | 43/2/0 | 129/5/0 | 68/2/0 | 122/1/0 | / | / |
| F3-5 | 23/0/0 | 286/1/0 | 95/1/0 | 152/6/0 | / | / |
| F3-6 | 60/4/0 | 159/3/0 | 515/11/0 | 311/12/0 | / | / |
| F3-7 | 28/2/0 | 112/2/1 | 769/17/0 | 442/5/1 | / | / |
| F3-8 | 23/3/2 | 228/0/0 | 53/4/0 | 105/6/1 | / | / |
| F3-9 | 19/0/0 | 179/0/0 | 83/3/0 | 58/3/0 | / | / |
| F3-10 | 38/1/0 | 78/0/0 | 121/4/0 | 49/3/0 | / | / |
| F3-11 | 9/2/0 | 115/2/1 | 63/4/0 | 93/4/0 | / | / |
| F3-12 | 20/3/0 | 298/58/0 | 65/2/0 | 386/102/1 | / | / |

All tested formulations showed consistent stability to freeze/thaw (FT) and H₂O₂ spiking stress conditions.

TABLE 15
SE-HPLC-1.

| | SE-HPLC (Monomer %/HMW %/LMW %) | | | | | | |
|---|---|---|---|---|---|---|---|
| Formulation | | 40° C. | | FT | | Agitation | |
| No. | T0 | 2 W | 4 W | 3 CYS | 5 CYS | 1 D | 3 D |
| F3-1 | 94.2/5.8/ | 90.5(↓3.7)/ | 87.0(↓7.2)/ | 93.6/6.4/ | 93.5(↓0.7)/ | 93.5/6.5/ | 92.8(↓1.4)/ |
| | 0.0 | 9.1/0.4 | 10.0/3.0 | 0.0 | 6.5/0.0 | 0.0 | 7.1/0.1 |
| F3-2 | 94.2/5.8/ | 91.1(↓3.1)/ | 87.9(↓6.3)/ | 93.7/6.3/ | 93.6(↓0.6)/ | 93.5/6.5/ | 93.0(↓1.2)/ |
| | 0.0 | 8.5/0.4 | 9.2/2.9 | 0.0 | 6.4/0.0 | 0.0 | 6.9/0.1 |
| F3-3 | 94.2/5.8/ | 91.1(↓3.1)/ | 88.0(↓6.2)/ | 93.7/6.3/ | 93.6(↓0.6)/ | 93.5/6.5/ | 93.0(↓1.2)/ |
| | 0.0 | 8.6/0.4 | 9.1/2.9 | 0.0 | 6.4/0.0 | 0.0 | 7.0/0.0 |
| F3-4 | 94.2/5.8/ | 91.3(↓2.9)/ | 88.1(↓6.1)/ | 93.8/6.2/ | 93.6(↓0.6)/ | 93.6/6.4/ | 93.1(↓1.1)/ |
| | 0.0 | 8.3/0.4 | 8.9/2.9 | 0.0 | 6.4/0.0 | 0.0 | 6.9/0.0 |
| F3-5 | 94.3/5.7/ | 91.5(↓2.8)/ | 88.4(↓5.9)/ | 93.9/6.1/ | 93.8(↓0.5)/ | 93.7/6.3/ | 93.3(↓1.0)/ |
| | 0.0 | 8.1/0.4 | 8.6/3.1 | 0.0 | 6.2/0.0 | 0.0 | 6.7/0.0 |
| F3-6 | 93.0/7.0/ | 89.7(↓3.3)/ | 86.4(↓6.6)/ | 92.1/7.9/ | 91.9(↓1.1)/ | 92.2/7.8/ | 91.6(↓1.4)/ |
| | 0.0 | 9.9/0.5 | 10.5/3.1 | 0.0 | 8.1/0.0 | 0.0 | 8.3/0.1 |
| F3- | 93.5/6.5/ | 90.8(↓2.7)/ | 87.5(↓6.0)/ | 92.9/7.1/ | 92.8(↓0.7)/ | 92.9/7.1/ | 92.5(↓1.0)/ |
| | 0.0 | 8.7/0.5 | 9.2/3.3 | 0.0 | 7.2/0.0 | 0.0 | 7.5/0.1 |
| F3-8 | 93.9/6.1/ | 91.1(↓2.8)/ | 87.9(↓6.0)/ | 93.5/6.5/ | 93.3(↓0.6)/ | 93.2/6.8/ | 92.7(↓1.2)/ |
| | 0.0 | 8.5/0.4 | 9.0/3.0 | 0.0 | 6.7/0.0 | 0.0 | 7.2/0.1 |
| F3-9 | 93.7/6.3/ | 90.4(↓3.3)/ | 87.2(↓6.5)/ | 93.1/6.9/ | 93.0(↓0.7)/ | 92.9/7.1/ | 92.4(↓1.3)/ |
| | 0.0 | 9.2/0.4 | 9.8/3.0 | 0.0 | 7.0/0.0 | 0.0 | 7.6/0.0 |
| F3-10 | 94.1/5.9/ | 90.9(↓3.2)/ | 87.5(↓6.6)/ | 93.6/6.4/ | 93.6(↓0.5)/ | 93.5/6.5/ | 93.0(↓1.1)/ |
| | 0.0 | 8.7/0.4 | 9.6/2.9 | 0.0 | 6.4/0.0 | 0.0 | 7.0/0.1 |
| F3-11 | 94.1/5.9/ | 90.1(↓4.0)/ | 86.4(↓7.7)/ | 93.6/6.4/ | 93.6(↓0.5)/ | 93.5/6.5/ | 92.9(↓1.2)/ |
| | 0.0 | 9.4/0.4 | 10.4/3.2 | 0.0 | 6.4/0.0 | 0.0 | 7.0/0.1 |
| F3-12 | 94.1/5.9/ | 89.6(↓4.5)/ | 85.8(↓8.3)/ | 93.8/6.2/ | 93.6(↓0.5)/ | 93.5/6.5/ | 92.9(↓1.2)/ |
| | 0.0 | 10.0/0.4 | 10.9/3.4 | 0.0 | 6.4/0.0 | 0.0 | 7.0/0.0 |

TABLE 16
SE-HPLC H₂O₂.

| Formulation | | | | | |
|---|---|---|---|---|---|
| No. | SE-HPLC (Monomer %/HMW %/LMW %) | | | | |
| (Excipient/ | | 0.1% (w/v) H₂O₂ | | 5 ppm H₂O₂ | |
| PS80 conc.) | T0 | 4 H | 24 H | 2 W | 4 W |
| F3-1 | 94.2/5.8/0.0 | 93.6/6.4/0.0 | 92.0(↓2.2)/7.9/0.1 | 91.6(↓2.6)/8.3/0.1 | 91.3(↓2.9)/8.6/0.1 |
| F3-2 | 94.2/5.8/0.0 | 93.7/6.3/0.0 | 92.5(↓1.7)/7.4/0.1 | 92.1(↓2.1)/7.8/0.1 | 91.8(↓2.4)/8.1/0.1 |

TABLE 17
iCIEF.

| | iCIEF (Main peak %/Acidic peaks %/Basic peaks %) | | | | | | |
|---|---|---|---|---|---|---|---|
| | | 40° C. | | FT | | Agitation | |
| Formulation No. | T0 | 2 W | 4 W | 3 CYS | 5 CYS | 1 D | 3 D |
| F3-1 | 58.5/32.5/ | 51.9/38.2/ | 45.0(↓13.5)/ | 54.5/32.4/ | 56.2/31.2/ | 53.9/35.9/ | 54.6(↓3.9)/ |
| | 9.1 | 9.9 | 43.0/12.0 | 13.1 | 12.6 | 10.1 | 32.9/12.5 |
| F3-2 | 58.9/31.3/ | 52.3/37.5/ | 45.3(↓13.6)/ | 55.8/30.8/ | 53.8(↓5.1)/ | 55.1/34.9/ | 56.1(↓2.8)/ |
| | 9.9 | 10.2 | 42.8/11.9 | 13.4 | 31.7/14.6 | 10.0 | 34.7/9.2 |
| F3-3 | 58.9/31.8/ | 51.4/38.4/ | 47.0(↓11.9)/ | 54.5/32.4/ | 55.6(↓3.3)/ | 58.8/33.2/ | 54.5(↓4.4)/ |
| | 9.3 | 10.2 | 42.7/10.3 | 13.1 | 33.7/10.6 | 8.0 | 32.7/12.8 |
| F3- | 57.8/33.7/ | 51.2/37.7/ | 46.0(↓11.8)/ | 54.9/32.4/ | 54.4(↓3.4)/ | 58.1/33.7/ | 56.0(↓1.8)/ |
| | 8.5 | 11.0 | 43.6/10.4 | 12.7 | 30.9/14.8 | 8.2 | 33.0/11.0 |
| F3-5 | 57.0/36.2/ | 51.8/37.7/ | 46.5(↓10.5)/ | 55.5/30.8/ | 51.9(↓5.1)/ | 58.7/33.0/ | 58.1/29.6/ |
| | 6.8 | 10.5 | 42.3/11.2 | 13.7 | 31.5/16.5 | 8.3 | 12.3 |
| F3-6 | 56.6/32.1/ | 51.1/38.2/ | 46.4(↓10.2)/ | 55.7/30.9/ | 55.8/33.0/ | 57.3/35.9/ | 53.7(↓2.9)/ |
| | 11.3 | 10.7 | 43.1/10.5 | 13.4 | 11.2 | 6.8 | 34.9/11.3 |
| F3-7 | 59.1/32.3/ | 52.2/36.7/ | 47.8(↓11.3)/ | 56.5/28.9/ | 55.1(↓4.0)/ | 57.0/32.7/ | 55.2(↓3.9)/ |
| | 8.7 | 11.1 | 40.8/11.4 | 14.6 | 31.0/13.9 | 10.3 | 31.5/13.3 |
| F3-8 | 56.6/34.7/ | 50.1/38.9/ | 45.5(↓1.1)/ | 57.4/29.7/ | 54.8(↓1.8)/ | 56.8/33.0/ | 58.7/28.7/ |
| | 8.7 | 11.0 | 42.7/11.8 | 12.8 | 33.0/12.2 | 10.2 | 12.6 |
| F3-9 | 57.6/31.9/ | 51.7/38.2/ | 45.9(↓1.7)/ | 56.1/30.0/ | 55.4(↓2.2)/ | 56.8/34.0/ | 54.0(↓3.6)/ |
| | 10.5 | 10.1 | 43.3/10.9 | 13.8 | 30.3/14.3 | 9.1 | 29.1/17.0 |
| F3-10 | 55.3/37.8/ | 50.5/38.4/ | 46.1(↓9.2)/ | 54.5/29.6/ | 56.2/32.3/ | 56.9/34.2/ | 55.2/30.9/ |
| | 6.9 | 11.0 | 43.6/10.3 | 15.9 | 11.5 | 8.9 | 13.9 |
| F3-11 | 56.0/31.6/ | 50.6/40.7/ | 44.0(↓12.0)/ | 53.9/28.7/ | 55.9/31.8/ | 58.0/33.8/ | 56.6/32.9/ |
| | 12.4 | 8.7 | 46.7/9.3 | 17.4 | 12.2 | 8.2 | 10.5 |
| F3-12 | 57.3/34.4/ | 48.5/42.2/ | 43.8(↓13.5)/ | 56.4/31.6/ | 54.6(↓2.7)/ | 57.0/33.5/ | 56.6/31.4/ |
| | 8.3 | 9.3 | 46.7/9.5 | 12.1 | 30.9/14.5 | 9.5 | 12.0 |

After freeze/thaw for up to 5 cycles and agitation for 3 days, the change in the main peak % as measured by iCIEF was minimal in all samples (+2.1%˜−5.1%), indicating the consistent freeze/thaw stability and agitation stability in all of the testing formulations.

TABLE 18
iCIEF H₂O₂ Study.

| | iCIEF (Main peak %/Acidic peaks %/Basic peaks %) | | | | |
|---|---|---|---|---|---|
| Formulation | | 0.1% (w/v) H₂O₂ | | 5 ppm H₂O₂ | |
| No. | T0 | 4 H | 24 H | 2 W | 4 W |
| F3-1 | 58.5/32.5/9.1 | 56.4(↓2.1)/35.1/ | 53.1(↓5.4)/37.3/ | 55.7(↓2.8)/34.8/ | 55.9(↓2.6)/35.1/ |
| | | 8.5 | 9.6 | 9.5 | 9.0 |
| F3-2 | 58.9/31.3/9.9 | 57.7(↓1.2)/34.6/ | 57.9(↓1.0)/34.2/ | 58.3(↓0.6)/33.9/ | 59.3/33.5/7.2 |
| | | 7.7 | 7.9 | 7.8 | |

After freeze/thaw for up to 5 cycles and agitation for 3 days, both non-reduced and reduced caliper-SDS testing data indicated that the purity % remained stable. When stored at 40° C. for 4 weeks, no substantial difference was observed in purity % by caliper-SDS-R for all formulations.

TABLE 19
Caliper-SDS-R.

| | Caliper-SDS-R (LC + HC)% | | | | | | | | | | |
|---|---|---|---|---|---|---|---|---|---|---|---|
| | | | | FT | | | | 0.1% (w/v) | | 5 ppm | |
| | | 40° C. | | 3 | 5 | Agitation | | H₂O₂ | | H₂O₂ | |
| Formulation No. | T0 | 2 W | 4 W | CYS | CYS | 1 D | 3 D | 4 h | 24 h | 2 W | 4 W |
| F3-1 | 99.7 | 99.5 | 99.4 | 99.6 | 99.6 | 99.2 | 99.5 | 99.6 | 99.6 | 99.5 | 98.9 |
| F3-2 | 98.8 | 99.6 | 98.4 | 99.5 | 99.6 | 99.5 | 99.3 | 99.6 | 99.5 | 99.5 | 99.6 |
| F3-3 | 99.6 | 99.5 | 99.3 | 99.3 | 99.6 | 99.6 | 99.6 | / | / | / | / |
| F3-4 | 99.6 | 99.5 | 99.4 | 99.6 | 99.6 | 98.8 | 99.5 | / | / | / | / |
| F3-5 | 99.5 | 99.4 | 99.3 | 99.6 | 99.6 | 99.0 | 99.1 | / | / | / | / |
| F3-6 | 99.4 | 99.5 | 99.4 | 99.1 | 99.6 | 99.3 | 99.6 | / | / | / | / |
| F3-7 | 99.6 | 99.4 | 99.5 | 99.6 | 99.5 | 99.5 | 99.6 | / | / | / | / |
| F3-8 | 99.3 | 99.5 | 99.4 | 99.6 | 99.5 | 99.5 | 99.6 | / | / | / | / |
| F3-9 | 99.5 | 99.3 | 98.3 | 99.5 | 99.6 | 99.6 | 99.6 | / | / | / | / |
| F3-10 | 99.6 | 99.5 | 99.2 | 99.3 | 99.6 | 99.4 | 99.6 | / | / | / | / |
| F3-11 | 99.5 | 99.3 | 99.2 | 99.6 | 99.6 | 99.5 | 99.5 | / | / | / | / |
| F3-12 | 99.4 | 99.6 | 99.5 | 99.5 | 99.6 | 99.6 | 99.5 | / | / | / | / |

TABLE 20
Caliper-SDS-NR.

| | Caliper-SDS-NR (purity)% | | | | | | | | | | |
|---|---|---|---|---|---|---|---|---|---|---|---|
| | | | | FT | | | | 0.1% (w/v) | | 5 ppm | |
| | | 40° C. | | 3 | 5 | Agitation | | H₂O₂ | | H₂O₂ | |
| Formulation No. | T0 | 2 W | 4 W | CYS | CYS | 1 D | 3 D | 4 h | 24 h | 2 W | 4 W |
| F3-1 | 98.0 | 95.6 | 94.0 | 98.0 | 97.6 | 98.4 | 98.1 | 97.8 | 96.1 | 98.3 | 98.5 |
| F3-2 | 98.0 | 96.7 | 95.7 | 98.3 | 98.0 | 97.9 | 97.6 | 97.8 | 97.3 | 98.2 | 98.4 |
| F3-3 | 98.2 | 97.2 | 96.5 | 98.3 | 98.4 | 98.3 | 98.1 | / | / | / | / |
| F3-4 | 98.2 | 97.2 | 96.1 | 98.1 | 98.3 | 97.9 | 98.1 | / | / | / | / |
| F3-5 | 98.0 | 97.2 | 95.8 | 98.0 | 98.2 | 98.0 | 98.2 | / | / | / | / |
| F3-6 | 98.1 | 97.0 | 95.9 | 98.1 | 98.3 | 97.5 | 98.1 | / | / | / | / |
| F3-7 | 98.0 | 95.4 | 95.7 | 98.5 | 98.1 | 98.0 | 98.3 | / | / | / | / |
| F3-8 | 98.1 | 97.0 | 95.9 | 98.3 | 98.3 | 98.0 | 98.2 | / | / | / | / |
| F3-9 | 98.1 | 96.5 | 96.4 | 98.4 | 98.2 | 98.6 | 98.2 | / | / | / | / |
| F3-10 | 97.6 | 97.2 | 95.9 | 98.3 | 98.3 | 98.1 | 98.1 | / | / | / | / |
| F3-11 | 98.2 | 95.4 | 96.1 | 97.4 | 97.7 | 98.2 | 98.0 | / | / | / | / |
| F3-12 | 98.3 | 95.7 | 96.1 | 98.0 | 98.1 | 98.2 | 98.3 | / | / | / | / |

TABLE 21
DSC.

| | T_{Onset} | Tₘ1 | Tₘ2 | Tₘ3 |
|---|---|---|---|---|
| Formulation No. | (° C.) | (° C.) | (° C.) | (° C.) |
| F3-1 | 60.6 | 68.8 | 74.3 | 79.2 |
| F3-2 | 60.3 | 69.0 | 74.2 | 79.1 |
| F3-3 | 60.5 | 69.1 | 74.4 | 79.3 |
| F3-4 | 60.4 | 68.7 | 74.2 | 79.1 |
| F3-5 | 60.2 | 68.7 | 74.2 | 79.1 |
| F3-6 | 58.1 | 66.0 | 72.0 | 76.9 |
| F3-7 | 56.7 | 65.5 | 71.9 | 76.8 |
| F3-8 | 57.9 | 67.2 | 73.0 | 78.1 |
| F3-9 | 58.8 | 67.2 | 73.1 | 78.2 |
| F3-10 | 60.7 | 68.9 | 74.3 | 79.2 |
| F3-11 | 60.2 | 69.5 | 74.2 | 79.0 |
| F3-12 | 60.4 | 68.8 | 74.2 | 79.1 |

### Example 2: Anti-TNFα Antibody 1 Formulation Confirmation

Anti-TNFα Antibody 1 molecule as well as its compatibility with the primary CCS were further evaluated in the formulation confirmation study. As denotated in the tables below, not tested is abbreviated as NT.

Samples were stored separately at different temperatures including 2-8° C. (inverted) for up to 9 months, 25° C. (inverted) for up to 3 months and 40° C. (upright) for 4 weeks. Among them, the inverted stability at 2-8° C. and 25° C. was to investigate the compatibility between the anti-TNFα Antibody 1 selected formulation (100 mg/mL anti-TNFα Antibody 1, 20 mM histidine buffer, 4.5% (w/v) sorbitol, 10 mM methionine, 0.02% (w/v) polysorbate 80 (PS80), pH 5.5) and the selected CCS. Testing items including appearance, pH, protein concentration, density, liquid particle counting, SE-UPLC, CE-SDS-NR & R, iCIEF and potency by ELISA were performed in this study. Appearances were visually inspected after sample pulling. pH, protein conc. and liquid particle counting were stored at 2-8° C. before analysis.

### Stability Study at 2-8° C. And 25° C.

Samples were slightly yellow, slightly opalescent, and free of visible particles after incubation at 2-8° C. for up to 9 months. Essentially free of visible particles were observed at 25° C. for 3 months. The amount of sub-visible particles (≥2 μm, ≥10 μm, and ≥25 μm) remained at very low level, compared with T0. Samples were also analyzed for pH and protein concentration.

TABLE 22
2-8° C./25° C.: appearance, protein
conc., pH and liquid particle counting.

| | | | | | | Sub-visible |
|---|---|---|---|---|---|---|
| | | | | | Protein | particles counts |
| Sampling | | | | | conc. | (#/mL, ≥2 μm/≥10 |
| point | | | Appearance | pH | (mg/mL) | μm/≥25 μm) |
| T0 | | | SY, SO, FP | 5.5 | 101.5 | 123/11/2 |
| 2-8° | C. | 2 W | SY, SO, FP | 5.5 | 99.8 | 114/15/0 |
| | | 4 W | SY, SO, FP | 5.5 | 99.7 | 167/12/0 |
| | | 3 M | SY, SO, FP | 5.6 | 99.3 | 18/0/0 |
| | | 6 M | SY, SO, FP | 5.6 | 100.0 | NT |
| | | 9 M | SY, SO, FP | 5.5 | 100.0 | NT |
| 25° | C. | 2 W | SY, SO, FP | 5.5 | 100.2 | 36/1/0 |
| | | 4 W | SY, SO, FP | 5.5 | 99.8 | 47/3/0 |
| | | 3 M | SY, SO, EFP* | 5.6 | 99.4 | 124/15/2 |

Slight decreases of 0.7% in monomer % by SE-UPLC and 5.9% in main peak % by iCIEF were found compared with T0, and no substantial change was observed in CE-SDS-R & NR when stored at 2-8° C. for 9 months. After incubation at 25° C. for 3 months, decreases of 1.4% in monomer % by SE-UPLC, 6.3% in main peak % by iCIEF compared with TO were observed and slight decreases of 1.0% and 1.4% in purity % by CE-SDS-R & NR were found, all within acceptable range for the accelerated conditions.

TABLE 23
2-8° C./25° C. SE-UPLC, iCIEF and CE-SDS-R & NR.

| | | | SE-UPLC | | | |
|---|---|---|---|---|---|---|
| Sampling | | | (Monomer %/ | iCIEF | CE-SDS-R | CE-SDS-NR |
| point | | | HMW %/LMW %) | (MP %/AP %/BP %) | (LC + HC)% | (Purity %) |
| T0 | | | 98.7/1.3/ND | 64.5/29.2/6.3 | 98.4 | 98.6 |
| 2-8° | C. | 2 W | 98.6/1.4/ND | 62.1/32.8/5.1 | 98.3 | 98.8 |
| | | 4 W | 98.5/1.5/ND | 62.5/31.8/5.7 | 97.8 | 98.5 |
| | | 3 M | 98.0/1.9/0.1 | 62.8/31.8/5.4 | 98.1 | 98.4 |
| | | 6 M | 97.8/2.1/0.1 | 60.4/33.6/5.9 | 98.1 | 98.2 |
| | | 9 M | 98.0/2.0/ND | 58.6/33.4/7.9 | 97.6 | 98.4 |
| 25° | C. | 2 W | 98.2/1.8/0.1 | 61.9/32.6/5.5 | 98.4 | 98.7 |
| | | 4 W | 97.8/2.1/0.1 | 61.6/32.3/6.1 | 97.9 | 98.3 |
| | | 3 M | 97.3/2.3/0.5 | 58.2/35.4/6.4 | 97.4 | 97.2 |

The potency results for samples stored at 2-8° C. for up to 9 months and 25° C. for up to 3 months are reported in Table 24. The relative potency values indicated no measurable decrease in potency by 9 months of incubation at 2-8° C. and 3 months at 25° C. compared with TO.

TABLE 24
2-8° C./25° C. ELISA.

Potency (%)

| | | | | |
|---|---|---|---|---|
| Storage condition | T0 | 3 M | 6 M | 9 M |

| | | | | | |
|---|---|---|---|---|---|
| 2-8° | C. | 106 | 106 | 107 | 115 |

| | | | |
|---|---|---|---|
| 25° | C. | 118 | NT |

### Stability Study at 40° C.

Samples remained slightly yellow, slightly opalescent, and free of visible particles with the continuous incubation at 40° C. for up to 4 weeks. The amount of sub-visible particles (≥2 μm, ≥10 μm, and ≥25 μm) remained at very low level. In addition, no substantial change in pH and protein conc. (100.0±10.0 mg/mL) was observed.

TABLE 25
40° C. appearance, protein concentration,
pH, and liquid particle counting.

| | | | | | Sub-visible |
|---|---|---|---|---|---|
| | | | | Protein | particles counts |
| Sampling | | | | conc. | (#/mL, ≥2 μm/≥10 |
| point | | Appearance | pH | (mg/mL) | μm/≥25 μm) |
| T0 | | SY, SO, FP | 5.5 | 101.5 | 123/11/2 |
| 40° C. | 2 W | SY, SO, FP | 5.5 | 100.2 | 17/2/0 |
| | 4 W | SY, SO, FP | 5.5 | 100.2 | 86/3/0 |

Following incubation at 40° C. for 4 weeks, decreases of 1.8% in monomer % by SE-UPLC and 10.9% in main peak % by iCIEF were observed, which were acceptable for the stress conditions (40° C.).

TABLE 26
40° C. SE-UPLC, iCIEF and CE-SDS-R & NR.

| | | SE-UPLC | | | |
|---|---|---|---|---|---|
| Sampling | | (Monomer %/ | iCIEF | CE-SDS-R | CE-SDS-NR |
| point | | HMW %/LMW %) | (MP %/AP %/BP %) | (LC + HC)% | (Purity %) |
| T0 | | 98.7/1.3/ND | 64.5/29.2/6.3 | 98.4 | 98.6 |
| 40° C. | 2 W | 97.5/2.1/0.4 | 56.3/36.1/7.6 | 97.7 | 97.8 |
| | 4 W | 96.9/2.4/0.7 | 53.6/36.4/10.0 | 96.5 | 95.9 |

The relative potency values showed no substantial decrease in potency after incubation at 40° C. for 4 weeks compared with TO.

TABLE 27
40° C. ELISA.

Potency (%)

| Storage condition | T0 | 4 W |
|---|---|---|
| 40° C. | 106% | 105% |

All samples exhibited an acceptable appearance profile after incubation at 25° C. for 3 months. Furthermore, the testing results also indicated no obvious effect on the stability of anti-TNFα Antibody 1 protein after incubation at 2-8° C. for 9 months.

Under accelerated and thermal stress conditions (25° C. and 40° C. incubation), the quality changes were all within the acceptable limit. The results of inverted stability at 2-8° C. incubation for up to 9 months and 25° C. incubation for 3 months indicated all the quality attributes were within the acceptable range and supported the selection of final CCS.

### Example 3: Preclinical Characterization of Anti-TNF a Antibody 1-Pharmacological Characteristics and Toxicology

This example evaluated the pharmacological characteristics of half-life extended TNFα antibody, in contrast to adalimumab.

It was observed that anti-TNFα antibody 1 has similar pharmacological characteristics to adalimumab, including TNFα binding and inhibition. Surface plasmon resonance (SPR) studies determined that anti-TNFα antibody 1 bound recombinant human TNFα with a comparable affinity to adalimumab (dissociation constant [KD]=0.081 and 0.079 nM for anti-TNFα antibody 1 and adalimumab, respectively). The EC₅₀ for in vitro inhibition of TNFα signaling as measured by inhibition of reporter luciferase activity driven by the transcription factor nuclear factor kappa B was roughly equivalent (18.17 ng/mL for anti-TNFα antibody 1 and 14.25 ng/ml for adalimumab), as was the IC₅₀ value (19.12 ng/ml for anti-TNFα antibody 1 and 13.52 ng/ml for adalimumab). Functional inhibition of TNFα was also assessed for IC₅₀ value based on the expression of CD62E in HUVEC cells and yielded comparable results (of 7.364 ng/ml for anti-TNFα antibody 1 and 7.468 ng/ml for adalimumab). In vitro inhibition of membrane-tethered TNFα (mTNFα) binding assessed in mTNFα target cells using flow cytometry similarly yielded comparable EC₅₀ results (0.04 μg/mL for anti-TNFα antibody 1 and 0.05 μg/mL for adalimumab). Similarly comparable EC₅₀ results were also observed in an ELISA multi-cycle kinetics analysis of assessing binding affinity for rhTNFα (0.015 μg/mL for anti-TNFα antibody 1 and 0.008 μg/mL for adalimumab).

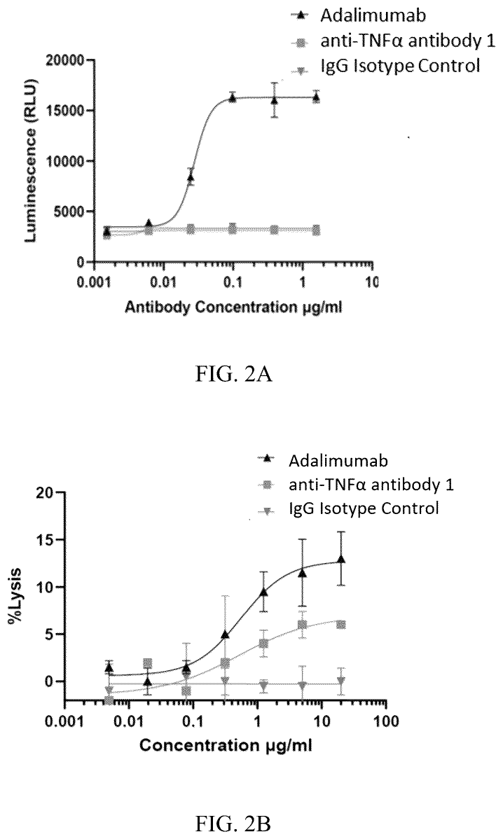

Consistent with low binding affinity for FcγRIIIA, anti-TNFα antibody 1 did not induce ADCC as compared to adalimumab. mTNFα target cells were incubated with test antibodies, followed by addition of ADCC effector cells expressing both FcγRIIIA and luciferase. Assessing luminescence demonstrated that, in contrast to adalimumab, anti-TNFα antibody 1 did not induce ADCC (FIG. 2A). Next, mTNFα target cells were incubated with a titration of test antibodies and normal human serum without and without heat inactivation. Anti-TNFα antibody 1 exhibited a lower binding affinity for C1q (EC₅₀=2.18 μg/mL) as compared to adalimumab and rituximab (EC₅₀ of 1.10 and 0.90 μg/mL, respectively). Consistent with low C1q binding affinity, anti-TNFαantibody 1 resulted in lower CDC than adalimumab (5-10% lysis with anti-TNFα antibody 1 and 10-15% lysis with adalimumab at antibody concentrations 5-20 μg/mL) (FIG. 2B).

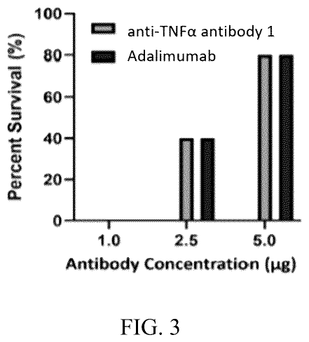

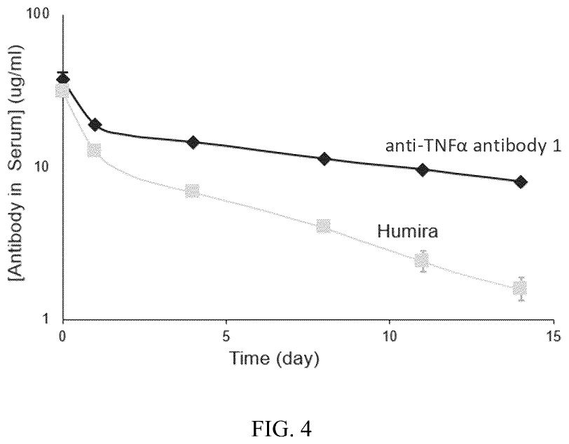

Additionally, anti-TNFα antibody 1 and adalimumab inhibited TNFα-induced lethality in a dose-dependent manner in D-galactosamine-sensitized mice at similar rates (FIG. 3). However, anti-TNFα antibody 1 has a higher fragment crystallizable receptor (“FcRn”) binding affinity as measured by SPR (K_{D}=6.54×10⁻⁸ M) compared to adalimumab (K_{D}=1.88×10⁻⁶ M), which extended anti-TNFα antibody 1 in vivo half-life (FIG. 4).

Species cross-reactivity assessments were determined by SPR using purified TNFα from human, cynomolgus monkey, mouse, rat, and rabbit (see Table 28), and supported monkey as the relevant toxicology species.

TABLE 28
Species cross reactivity to TNFα.

| Species Cross Reactivity to TNFα | K_{D} (M) |
|---|---|
| Human | 4.09 × 10⁻¹¹ |
| Cynomolgus monkey | 7.24 × 10⁻¹¹ |
| Mouse | 4.68 × 10⁻⁹ |
| Rat | 3.82 × 10⁻⁸ |
| Rabbit | 3.17 × 10⁻⁸ |

In the human neonatal FcRn expressed transgenic mice, the pharmacokinetics profile of anti-TNFα antibody 1 was found to be relatively similar to that of adalimumab with the exception of an extended (≥2-fold longer) half-life (t_{1/2}=9.9 days and 4.4 days for anti-TNFα antibody 1 and adalimumab, respectively Table 29).

TABLE 29
PK Parameters following a single IV dose in mice.

| | Cₘₐₓ | AUC_{inf} | t_{1/2} |
|---|---|---|---|
| Treatment | (μg/mL) | (μg · day/mL) | (day) |
| Adalimumab | 28.7 (2.1) | 101.4 (13.5) | 4.4 (0.6) |
| anti-TNFα antibody 1 | 35.9 (7.4) | 301.1 (36.3) | 9.9 (1.6) |

Anti-TNFα antibody 1 by once weekly subcutaneous administration for 13 weeks in cynomolgus monkeys demonstrated no adverse safety findings at any dose tested up to 200 mg/kg, providing an adequate therapeutic window for assessment in humans. Further nonclinical safety studies were assessed in cynomolgus monkey including both a single-dose study and 13-week repeat dose study with Q1W SC administration followed by a 4-week recovery period (Tables 30 and 31).Antidrug antibodies to anti-TNFα antibody 1 were detected in 7 animals from all groups ( 1/10, 2/10, 3/10, and 1/10 at 0, 30, 80, and 200 mg/kg, respectively). Most ADA-positive samples (4/7 animals) had low titers and they were only detectable predose on Day 1.

TABLE 30
PK results from single administration of anti-
TNFα antibody 1 to cynomolgus monkeys.

| Dose Route | IV Infusion | SC Injection | | |
|---|---|---|---|---|
| Dose Level (mg/kg) | 30 | 10 | 30 | 100 |
| Cₘₐₓ (μg/mL) | 867 | 110 | 326 | 1210 |
| Tₘₐₓ (h) | 1.17 | 112 | 68.0 | 64.0 |
| T_{1/2} (h) | 181 | 136 | 213 | 253 |
| Vdₛₛ (L/kg) | 0.0557 | — | — | — |
| Cl (mL/min/kg) | 0.00357 | — | — | — |
| AUCₗₐₛₜ (μg · h/mL) | 101,000 | 21,000 | 76,900 | 356,000 |
| Bioavailability (%) | — | 62.4 | 76.1 | 106 |

TABLE 31
PK results from once weekly administration of
anti-TNFα antibody 1 to cynomolgus monkeys.

| Dose | Study | | Cₘₐₓ | | AUC_{0-168 h} |
|---|---|---|---|---|---|
| (mg/kg) | Day | Sex | (μg/mL) | Tₘₐₓ (h) | (h · μg/mL) |
| 30 | 1 | Male | 331 | 96.0 | 48,600 |
| | | Female | 327 | 48.0 | 48,700 |
| | 92 | Male | 752 | 8.0 | 118,000 |
| | | Female | 814 | 24.0 | 117,000 |
| 80 | 1 | Male | 875 | 96.0 | 126,000 |
| | | Female | 865 | 48.0 | 125,000 |
| | 92 | Male | 1340 | 24.0 | 185,000 |
| | | Female | 1470 | 24.0 | 190,000 |
| 200 | 1 | Male | 2040 | 48.0 | 267,000 |
| | | Female | 1790 | 48.0 | 245,000 |
| | 92 | Male | 2500 | 24.0 | 327,000 |
| | | Female | 2670 | 24.0 | 381,000 |

Anti-TNFα antibody 1 was well tolerated following a single-dose SC administration at 0, 30, 100, or 300 mg/kg in cynomolgus monkey. No TNFα antibody 1-related adverse findings were observed in the 13-week repeat-dose SC toxicity study with a 4-week recovery period in cynomolgus monkey. Anti-TNFα antibody 1 was administered via SC injection at 0, 30, 80, or 200 mg/kg Q1W for 13 weeks. No off-target binding of anti-TNFα antibody 1 was observed. The no-observed-adverse-effect-level (NOAEL) was 200 mg/kg, the highest dose evaluated in the study.

TABLE 32
Anti-TNFα antibody 1 toxicology studies.

| | MTD | NOAEL | |
|---|---|---|---|
| Study Type | (mg/kg) | (mg/kg) | Observations |
| Tissue cross-reactivity | NA | NA | No off-target binding in either human or |
| of anti-TNFα antibody 1 | | | cynomolgus monkey tissue |
| Anti-TNFα antibody 1 | 300 | NA | No anti-TNFα antibody 1-related changes |
| single SC dose at 30, | | | noted in clinical observations (including |
| 100, and 300 mg/kg + | | | injection site observation), body weight, food |
| 28-day recovery; 4 | | | consumption, serum chemistry, coagulation, |
| monkeys/sex/dose | | | urinalysis, or gross and histopathology |
| | | | Minor changes in reticulocytes on Day 2 at |
| | | | 300 mg/kg, recovered at the end of 28 days |
| Anti-TNFα antibody 1 | NA | 200 | No anti-TNFα antibody 1-related changes |
| repeat SC dose at 0, 30, | | | noted in clinical observations (including |
| 80, or 200 mg/kg once | | | injection site observation), body weight, food |
| weekly for 13 weeks + | | | consumption, ophthalmic examinations, body |
| 4 weeks recovery; 5 | | | temperature, safety pharmacology, |
| monkeys/sex/dose-level | | | hematology, serum chemistry, coagulation, |
| | | | urinalysis, gross pathology, organ weights, or |
| | | | histopathology |

A maximum safe starting dose for the first in human (FIH) study of anti-TNFα antibody 1 was calculated from the in cynomolgus monkeys by scaling to the human-equivalent dose based on body surface area, and then adjusting for a 10-fold safety margin. Applying this method to anti-TNFα antibody 1 provided a maximum recommended starting dose (MRSD) of 6.4 mg/kg administered no more than weekly: calculated from the 1/10th of the NOAEL of 200 mg/kg/dose determined in cynomolgus monkeys, MRSD was 1200 mg (20 mg×60 kg human). The starting dose of 20 mg SC single dose (approximately 0.27-0.45 mg/kg for participants weighing 45 to 75 kg) and the planned maximum dose of 240 mg SC single dose (approximately 3.2 to 5.4 mg/kg for participants weighing 45 to 75 kg) fell below the MRSD. In addition, exposure-based safety margins were estimated using the TK data at the NOAEL in monkey and the projected anti-TNFα antibody 1 systemic exposure (AUC_{inf}) for the proposed clinical dose range of 20-240 mg SC estimated based on published adalimumab PK parameters (Dillingh M R, Reijers J A, Malone K E, et al. Clinical Evaluation of Humira® Biosimilar ONS-3010 in Healthy Volunteers: Focus on Pharmacokinetics and Pharmacodynamics. Front Immunol. 2016; 7:508) (Table 33). Calculations assumed linear PK and a two-fold higher half-life for anti-TNFα antibody 1 compared to adalimumab, based on animal anti-TNFα antibody 1 PK data. Using this approach, the 200 mg/kg dose level in the 13-week anti-TNFα antibody 1 monkey toxicity study provided approximately 125- and 10-fold safety margins for the doses of 20 mg (starting dose) and 240 mg (maximum dose), respectively (Table 33).

TABLE 33
Predicted anti-TNFα antibody 1 PK parameters
and safety margin calculated for clinical dose.

| | | | | anti-TNFα antibody 1 observed |
|---|---|---|---|---|
| | | anti-TNFα antibody 1 | | cynomolgus monkey values |
| | Adalimumab | predicted clinical values | | in 13-week study |
| Dose | 40 mg | 20 mg | 240 mg | 200 mg/kg/week |
| AUC_{inf} | 2,631 | 2,631 | 31,573 | 327,000 |
| (hr · μg/mL) | | | | |
| CL (mL/hr) | 15.2 | 7.6 | 7.6 | — |
| Safety margin | — | 125-fold | 10-fold | — |

### Example 4: Dosage Escalation of Anti-TNFα Antibody 1 in Healthy Human Volunteers

This Example describes a Phase 1, double-blind, randomized, placebo-controlled single ascending dose (SAD) study that evaluated the safety, tolerability, and PK profile of anti-TNFα antibody 1 in healthy volunteers.

In total, forty-eight (48) healthy volunteers were enrolled across 6 cohorts across the varying dosing levels of 20 mg, 40 mg, 80 mg, 160 mg, and 240 mg. In each cohort, 8 healthy volunteers were randomized 3:1 to of anti-TNFα antibody 1 or placebo, thus a total of 36 participants received of anti-TNFα antibody 1 as a single subcutaneous (SC) injection. Cohort 6 was added to this study via amendment to repeat evaluation of the 40 mg dose level from Cohort 2. This was done to confirm that the PK profile of 40 mg dose is consistent with expectations based on the other dose levels evaluated.

Participants were randomized 3:1 to anti-TNFα antibody 1 or placebo per cohort. For each cohort, 2 sentinel participants (1 anti-TNFα antibody 1 and 1 placebo) were dosed and observed for 72 hours (observation postdose through Day 2 assessments inpatient followed by an outpatient visit at 72 hours postdose). Following the observation period, and approval by the Investigator, the remaining participants in that cohort received a single SC dose of anti-TNFα antibody 1 or placebo. Escalation to the next cohort commenced after review of the 2-week safety and available PK data by the SRC. This process was repeated for all subsequent cohorts. All participants completed dosing and 5 of 6 cohorts, including the top dose of 240 mg, completed all follow-up visits.

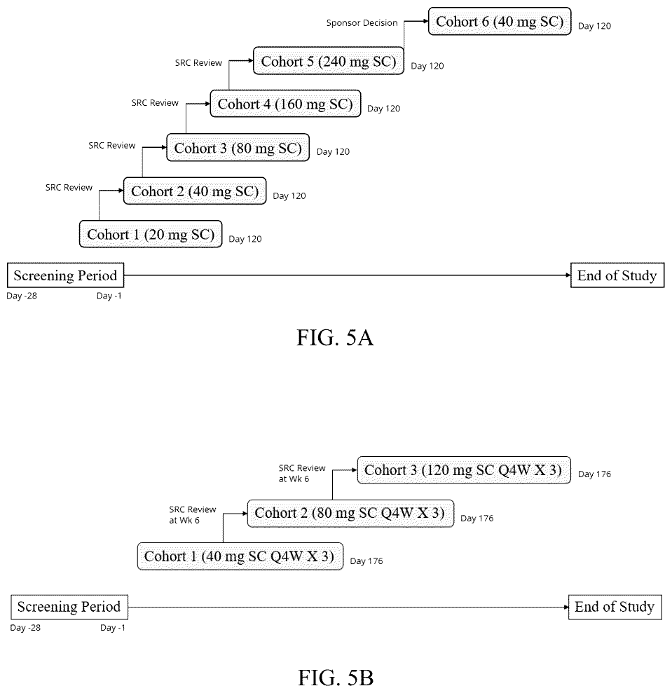

Each participant received a single SC dose of anti-TNFα antibody 1 or placebo, according to the dose escalation scheme described in FIG. 5A and observed for 120 days.

Participants were admitted into the Phase 1 clinical research unit (CRU) on Day-1 and were discharged on Day 2 after sample collection and study assessments. Participants returned to the CRU for scheduled follow-up visits on the days specified in Table 34.

The decision to escalate dose was based on the review of safety and available PK data by the SRC. At a minimum, the decision regarding dose escalation was based on at least 7 participants per cohort who have either completed the Day 15 visit or terminated early due to a DLT or anti-TNFα antibody 1-related AE.

TABLE 34
Schedule of Assessments

| | Timepoints Relative to Dose Administration | | | | | | | | | | | | | | | |
|---|---|---|---|---|---|---|---|---|---|---|---|---|---|---|---|---|
| | Screening | | | D 2 | D 3 | D 4 | D 5 | D 8 | D 15 | D 22 | D 29 | D 43 | D 57 | D 71 | D 92 | D 120 |
| | D −28 | | | (24 ± | (48 ± | (72 ± | (96 ± | (168 ± | (±1 | (±1 | (±1 | (±2 | (±2 | (±3 | (±3 | (±3 D)/ |
| | to D −2 | D −1 | D 1 | 1 h) | 12 h) | 3 h) | 4 h) | 4 h) | D) | D) | D) | D) | D) | D) | D) | ET |
| Informed Consent | x | | | | | | | | | | | | | | | |
| Medical | x | | | | | | | | | | | | | | | |
| History/ | | | | | | | | | | | | | | | | |
| Demographics | | | | | | | | | | | | | | | | |
| Inclusion/Exclusion | x | x | | | | | | | | | | | | | | |
| Randomization/ | | | | x | | | | | | | | | | | | |
| Study Drug | | | | | | | | | | | | | | | | |
| Administration | | | | | | | | | | | | | | | | |
| Concomitant | x | x | x | x | x | x | x | x | x | x | x | x | x | x | x | x |
| Medications and | | | | | | | | | | | | | | | | |
| Procedures | | | | | | | | | | | | | | | | |
| Physical | x | x | | | | | x | | | | | | x | | | x |
| Examination | | | | | | | | | | | | | | | | |
| Chest X-ray | x | | | | | | | | | | | | | | | |
| HBV, HCV, HIV, | x | | | | | | | | | | | | | | | |
| TB, COVID-19 | | | | | | | | | | | | | | | | |
| Testing | | | | | | | | | | | | | | | | |
| ECG (12-Lead) | x | | x | | | x | x | x | | | | | x | | | x |
| Vital Signs | x | x | x | x | x | x | x | x | x | x | x | x | x | x | x | x |
| Urinalysis | x | | x | | | | | | | | | | | | | x |
| AE Assessment | x | x | x | x | x | x | x | x | x | x | x | x | x | x | x | x |
| Blood Chemistry, | x | | x | x | | | | x | | x | | x | | x | x | x |
| Hematology, | | | | | | | | | | | | | | | | |
| Coagulation | | | | | | | | | | | | | | | | |
| PK Blood Sampling | | | x | x | x | x | x | x | x | x | x | x | x | x | x | x |
| Blood Sample for | | | x | | | | | | | x | | | x | | | x |
| ADA | | | | | | | | | | | | | | | | |
| PD/Biomarker | | | x | x | x | x | x | x | x | x | x | x | x | x | x | x |
| Sampling | | | | | | | | | | | | | | | | |

### Inclusion Criteria

Participants were selected for treatment based on the following criteria:
1) Healthy male or female participants 18 to 55 years of age at Screening.
2) Body weight ≥50 kg for male participants and ≥45 kg for female participants; body mass index of 18 to 35 kg/m² for both male and female participants.
3) Considered in good health as determined by the Investigator.
   Good health of the HV includes the absence of significant cardiac, pulmonary, hepatic, renal, hematologic, gastrointestinal, nervous system, psychiatric, metabolic abnormalities, clinically significant infections, coagulopathies, and severe allergies, as determined by the results of physical exam (PE), detailed previous medical history, vital signs, 12-lead ECG, chest X-ray, and clinical laboratory tests.
4) Female participants of child-bearing potential
5) Male participants must be surgically sterile for ≥12 weeks at the time of Screening or agree to use effective contraception as described in the Section 9.2 from the signing of the informed consent to 6 months after the last scheduled dose of the study drug. Male participants must also agree not to donate sperm during this timeframe.
6) Willing and able to understand the characteristics and purposes of the study, including possible risks involved, and willing to comply with all the study requirements and provide written informed consent for the study.

### Exclusion Criteria

Eligible participants did not meet any of the following criteria:
1) Surgery within 4 weeks before Screening or planned surgery during the clinical study.
2) Use of prescription medications, biological products, or other medicines within 2 weeks before Study Day 1 or 5 half-lives of the product, whichever is greater. Use of over-the counter medications or vitamins/dietary supplements within 7 days of dosing unless considered by the Investigator to not pose a risk or impact the study results.
3) Treatment with any investigational drug within 30 days or 5 half-lives, whichever is greater, before the first dose of the study drug, or currently enrolled in another clinical study.
4) Previously received an anti-TNF antibody or fusion protein medication.
5) Clinically significant ECG abnormality, such as a QTcF (QT interval corrected by Fridericia's formula) interval >450 ms for male participants or >470 ms for female participants.
6) Positive for HIV infection, active hepatitis C, or hepatitis B (surface antigen positive or core antibody positive with surface antibody negative).
7) Positive for COVID-19 virus according to local practice.
8) Positive QuantiFERON®-TB Gold or T-SPOT® test (only one of these tests must be done) for Mycobacterium tuberculosis.
9) Bacteria, viruses, systemic fungi, parasites, or other opportunistic infections within 30 days before Study Day 1 with the exception of minor nasopharyngitis that has resolved >1 week prior to dosing. Expected hospitalization or injection of antibiotics within 3 months before using the study drug.
10) Documented history of drug or alcohol abuse in the previous 12 months before Screening, or positive urine drug screen on Screening and/or Day −1.
11) Donated blood (including component blood) or lost >400 mL within 3 months before Screening or received a transfusion within 3 months of Screening.
12) History of relevant allergies (including allergy to any murine or human-derived protein or immunoglobulin products, rubber or latex, or other allergies that in the opinion of the investigator make inclusion in the study inappropriate).
13) Average daily smoking >10 cigarettes or cigarette equivalents per day within 6 months of Screening.
14) Consume >14 standard units of alcohol per week (1 standard unit is equivalent to approximately 360 mL of beer, 45 mL of spirits with 40% alcohol, or 150 mL of wine), or a positive alcohol breath test on Day-1.
15) Positive serum pregnancy test during Screening or positive urine pregnancy test at Day-1.
16) Women who are planning to become pregnant, breastfeeding, or planning to breastfeed at any time between signing of the informed consent and 6 months after the last dose of the study drug.
17) Clinically significant blood chemistry abnormality at Screening, as indicated by alanine aminotransferase (ALT) ≥1.5× upper limit of normal (ULN), aspartate aminotransferase
18) (AST) ≥1.5×ULN, total bilirubin ≥1.5×ULN, direct bilirubin ≥1.5x ULN, or serum creatinine ≥ ULN.
19) Clinically significant hematologic abnormality at Screening as indicated by white blood cell count <2500/μL (2.5×10⁹/L), hemoglobin <10 g/dL (100 g/L), or platelet count <75,000/μL (75×10⁹/L).
20) History of any malignancy within 5 years, except for successfully treated nonmelanoma skin cancer or localized carcinoma in situ of the cervix.
21) Employees or related personnel of the clinical research unit (CRU), the Sponsor, or the contract research organization.
22) Any condition that might interfere with the study assessments or the safety evaluation of the investigational product.
23) Any condition or other reason that, in the opinion of the Investigator, will make the participant not appropriate for enrollment or may prevent the participant from completing the study.

### Safety Analyses and Endpoints

Treatment-emergent adverse events (TEAEs), serious adverse events (SAEs), laboratory assessments, and electrocardiogram (ECG) were used as primary endpoints of the study. An adverse event (AE) was any unfavorable and unintended sign (including an abnormal laboratory finding), symptom, or disease temporally associated with the use of a medical treatment or procedure that may or may not be considered related to the medical treatment or procedure. An AE is a term that is a unique representation of a specific event used for medical documentation and scientific analyses.

A TEAE is an AE that emerges or worsens in the period after dosing. Participants were instructed to report all AEs and were asked a general health status question at each study visit.

All AEs were reviewed and assessed as serious or nonserious. AEs were assigned the following attributes:
AE diagnosis or syndrome(s) if known
   If not known at time of the report, record the signs and/or symptoms as AEs and provide an updated report with diagnosis when obtained.
Dates of onset and resolution
Severity as defined per protocol
Assessment of relatedness to anti-TNFα antibody 1

In general, an AE that was the primary cause of subsequent events was identified by the primary cause (e.g., for dehydration due to diarrhea, the AE would be diarrhea). However, AEs occurring secondary to an initiating event that are separated in time were recorded as independent events (e.g., for sepsis secondary to pneumonia, both events were recorded).

### Grades of AE

Grade refers to the severity of the AE. The CTCAE displays Grades 1 through 5 with unique clinical descriptions of severity for each AE based on this general guideline:
Grade 1: mild; asymptomatic or mild symptoms; clinical or diagnostic observations only; intervention not indicated.
Grade 2: moderate; minimal, local, or noninvasive intervention indicated; limiting age-appropriate instrumental ADL*.
Grade 3: severe or medically significant but not immediately life-threatening;
hospitalization or prolongation of hospitalization indicated; disabling; limiting self-care ADL**.
Grade 4: life-threatening consequences; urgent intervention indicated.
Grade 5: death related to AE.

Adverse events (AEs) were summarized by study part and dose, if applicable.

Secondary endpoints included serum concentration of anti-TNFα antibody 1 and derived PK parameters:
maximum observed serum concentration (Cₘₐₓ)
time at which the Cₘₐₓ is observed (Tₘₐₓ)
area under the serum concentration-time curve from time 0 extrapolated to infinity (AUC_{inf})
area under the serum concentration-time curve from time 0 to the last quantifiable concentration (AUCₗₐₛₜ)
terminal half-life (t_{1/2})
apparent clearance (CL/F) following extravascular dosing
apparent volume of distribution (V_{z}/F) following extravascular dosing.

### Serious Adverse Event

An SAE is any untoward medical occurrence that at any dose,
Results in death
Is immediately life-threatening
Requires hospitalization or prolongation of existing hospitalization
   Elective or pre-planned treatment for a pre-existing condition that is unrelated to the indication under study and has not worsened since signing the ICF (as documented as medical history on the eCRF) is not considered an SAE.
   Scheduled therapy for the target disease of the study, including admissions for transfusion support or convenience, is not considered an SAE.
Results in persistent or significant disability/incapacity
Results in congenital anomaly/birth defect
Is considered an important medical event
   If an AE does not meet one of the criteria for seriousness but the Investigator or Sponsor considers the event to be clinically important, the event could be classified as an SAE under the criterion of “Important medical event.” Examples of such medical events may include allergic bronchospasm requiring intensive treatment in an emergency department or at home; blood dyscrasias; or convulsions that do not result in hospitalization.

### Abnormal Laboratory and Electrocardiogram Values

The Investigator reviewed clinical laboratory tests and ECG results and determine whether an abnormal value represents a clinically significant change from the participant's baseline value for which an AE were reported.

### Example 5: Anti-TNFα Antibody 1 Safety, PK, and PD Analysis

This example describes safety, PK, and PD results and analysis from the FIH study of Example 4.

### Results of Safety Evaluations

All participants completed dosing and 5 of 6 cohorts, including the top dose of 240 mg, completed all follow-up visits. Cohort 6 performed to repeat evaluation of the 40 mg dose level from Cohort 2. This was done to confirm that the PK profile of 40 mg dose was consistent with expectations based on the other dose levels evaluated. SRC meetings were conducted when there were 2 weeks of safety data available for cohorts 1 through 4 as the basis of decisions regarding dose escalation.

One case of serious infection was observed: a perianal abscess considered grade 3 and related to study drug in Cohort 6 (40 mg). Serious infections are a potential risk for anti-TNFα antibody 1 and anticipated for the class of anti-TNFα antibodies at an incidence of 2 to 4 events per 100 patient-years. The number and duration of exposures for anti-TNFα antibody 1 is not yet adequate to characterize an incidence rate; however, there is no indication that the incidence or severity of serious infections for anti-TNFα antibody 1 is beyond that of the class.

Other than the serious infection, the adverse events in the SAD study were mild or moderate. The incidence of TEAEs was similar between participants administered anti-TNFα antibody 1 (80.6%) and participants administered placebo (83.3%) and was not related to anti-TNFα antibody 1 dose. There were no cohort-related trends in chemistry, hematology, urinalysis, vitals or ECGs. The subcutaneous injection was well-tolerated with only mild injection site reactions or erythema. From preliminary blinded combined cohort data (20 to 240 mg SC), the most frequent AEs were upper respiratory tract injection (19 of 48; 40%), headache (9; 19%), injection site erythema and ligament strain (each with 4; 8%), arthralgia, and COVID-19 (each with 3; 6%).

Overall, the blinded single dose profile of anti-TNFα antibody 1 up to 240 mg is consistent with other anti-TNFα antibodies.

### Pharmacodynamic and Pharmacokinetic Results

Following a single SC dose of anti-TNFα antibody 1, serum PK parameters were assessed (Table 35). Following a single subcutaneous administration of anti-TNFα antibody 1 at 20 to 240 mg, generally similar absorption of anti-TNFα antibody 1 was observed for all doses, with median Tₘₐₓ ranging from of 120.27 hours (5 days) postdose at 20 mg anti-TNFα antibody 1 to 337.83 hours (14 days) postdose at 240 mg anti-TNFα antibody 1. After reaching Cₘₐₓ, anti-TNFα antibody 1 concentrations declined, with mean t1/2 values ranging from 13.3 to 69.9 days with values generally increasing with the increase in anti-TNFα antibody 1 dose. Anti-TNFα antibody 1 Cₘₐₓ increased in a dose-proportional manner while AUCₗₐₛₜ and AUC_{inf} tended to increase in a slightly greater than dose-proportional manner over the dose range from 20 to 240 mg.

Following a single subcutaneous administration of anti-TNFα antibody 1 at 40, 80, 160, or 240 mg, mean ex vivo-stimulated TNFα and IFN-γ concentrations decreased with maximum TNFα percentage change from baseline approaching-100% and maximum IFN-γ percentage change from baseline of −71%, −53%, −70%, and −62%, respectively. After reaching a maximum decrease, IFN-γ returned to baseline by approximately 42 to 56 days postdose, while TNFα at anti-TNFα antibody 1 40 (Cohort 6), 80, 160, or 240 mg did not return to baseline during the 120-day collection period.

Data from Cohort 2, the first 40 mg cohort, appeared to be aberrant with concentrations much closer to that of Cohort 1 than anticipated. Due to this, the dose level of Cohort 2 (40 mg) was dosed in a repeat cohort (Cohort 6, 40 mg Repeat). Data from Cohort 6 showed a profile that was consistent with data from Cohorts 1, 3, 4, & 5, and Cohort 2 PK was considered aberrant from unknown etiology.

TABLE 35
Serum PK parameters of anti-TNFα antibody 1.

| | anti-TNFα antibody 1 Geometric Mean (Geometric CV %) | | | | | |
|---|---|---|---|---|---|---|
| | 20 mg | 40 mg | 40 mg Repeat | 80 mg | 160 mg | 240 mg |
| Parameter | (N = 6) | (N = 6) | (N = 6) | (N = 6) | (N = 6) | (N = 6) |
| Cₘₐₓ (μg/mL) | 2.20 | 2.53 | 4.45 | 10.0 | 16.9 | 28.1 |
| | (44.4) | (37.1) | (35.0) | (39.8) | (32.4) | (54.9) |
| Tₘₐₓ (h) ^{a} | 120.27 | 253.84 | 168.20 | 168.18 | 167.94 | 337.83 |
| | (48.02-671.17) | (97.05-575.40) | (71.68-674.22) | (48.00-311.77) | (96.12-336.22) | (47.95-671.12) |
| AUCₗₐₛₜ | 2510 | 2850 | 5640 | 14,800 | 29,000 | 49,300 |
| (μg · h/mL) | (38.5) | (67.1) | (44.4) | (32.4) | (22.2) | (40.8) |
| AUC_{inf} | 2560 | 2820 | 6210 | 15,200 | 32,100 | 49,200 |
| (μg · h/mL) | (39.0) | (79.5) | (50.3) | (37.8) | | |
| t_{1/2} | 13.3 | 18.5 | 23.5 | 45.2 | 69.9 | 64.0 |
| (days) | (81.1) | (99.9) | (83.2) | (31.9) | (21.1) | (24.5) |
| CL/F | 0.00780 | 0.0142 | 0.00644 | 0.00528 | 0.00498 | 0.00488 |
| (L/h) | (39.0) | (79.5) | (50.3) | (37.8) | | |
| V_{z}/F | 3.60 | 7.44 | 5.24 | 7.11 | 9.26 | 7.82 |
| (L) | (40.1) | (33.2) | (37.7) | (32.2) | | |

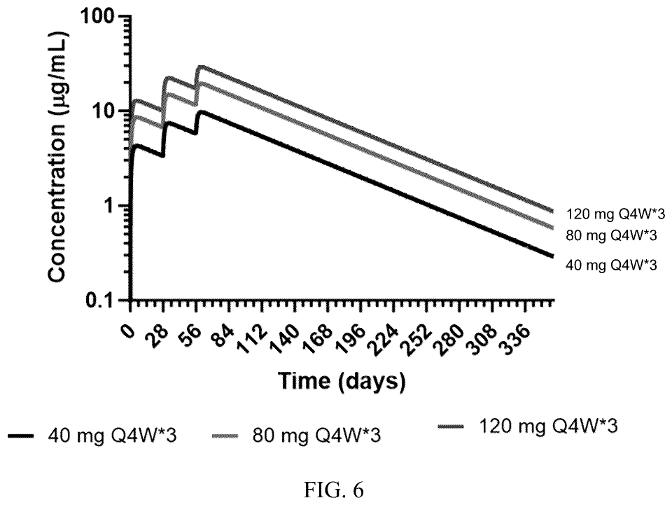

Predictive simulated dose concentrations for the MAD study are shown in FIG. 6. The PK of anti-TNFα antibody 1 was determined to be best characterized by a one-compartment model with a first-order absorption (with a lag time) and linear elimination, as evidenced by goodness of fit, and the precision of parameter estimation. Inter-individual variability parameters were included on CL/F, V/F and absorption rate constant. The combined proportional and additive error was employed as the residual error model. As demonstrated by a percent relative standard error (% RSE), all parameters were estimated (Table 36).

TABLE 36
Anti-TNFα antibody 1 PopPK model parameter estimates

| | | | RSE | Shrinkage |
|---|---|---|---|---|
| Parameters | Parameters Description | Estimate | (%) | (%) |
| θ₁ | CL/F (L/hr) | 0.00406 | 4.2 | — |
| θ₇ | BWT on CL/F | 0.662 | 30.5 | — |
| θ₂ | V/F (L) | 8.66 | 4.9 | — |
| θ₈ | BWT on V/F | 0.665 | 39.8 | — |
| θ₃ | Kₐ (1/hr) | 0.028 | 16.4 | — |
| θ₄ | ALAG1 (hr) | 2.05 | 15.7 | — |
| η₁ | η_{CL} (%) | 18.2 | 14.4 | 9.5 |
| η₂ | ηᵥ (%) | 27.3 | 11.6 | 2.5 |
| η₃ | η_{Ka} (%) | 96.8 | 11.2 | 1.1 |
| θ₅ | Proportional residual | 0.106 | 10.0 | — |
| | error (%) | | | |
| θ₆ | Additive residual | 191.8 | 18.7 | — |
| | error (ng/mL) | | | |

A prediction-corrected visual predictive check (pc-VPC) was produced to check the potential bias in model predictions by accounting for systematic differences between the observed and predicted data. Simulations of 500 datasets were performed. The observed and simulated data were normalized by the typical population prediction for the median independent variable in the bin. For each simulation, the 10th, 50th (median), and 90th prediction intervals were calculated. The 95% confidence intervals were calculated for each of the prediction intervals and overlaid with the 10th, 50th (median), and 90th intervals of the observed data. The post-hoc estimated median half-life of anti-TNFα antibody 1 was determined using this model as 60.9 days, an extension of almost 5x compared to adalimumab.

### Example 6: Multiple Ascending Dose Study in Participants with Rheumatoid Arthritis (RA)

This example describes a Phase 1, double-blind, randomized, placebo-controlled, multiple ascending dose (MAD), safety, tolerability, and PK study of anti-TNFα antibody 1 in participants with RA. Participants are organized into 3 cohorts, each participant receiving multiple doses of anti-TNFα antibody 1 or placebo, according to the dose escalation scheme (FIG. 5B).
Cohort 1:40 mg SC Q4W x 3 administrations (40 mg Q4W*3).
Cohort 2:80 mg SC Q4W x 3 administrations (80 mg Q4W*3).
Cohort 3:120 mg SC Q4W x 3 administrations (120 mg Q4W*3).

The decision to escalate the dose is based on the review of safety and available PK data. Predictive simulated dose concentrations for the MAD study are shown in FIG. 6. The Tₘₐₓ for anti-TNFα antibody 1 is expected to be reached on or before Day 15 based on median Tₘₐₓ estimates from the SAD part of the study; therefore, a safety review takes place 15 days after the second administration of study drug for all participants in a given cohort. Singles doses from 20 to 240 mg were studied with all dose levels being safe and well tolerated. Participants are followed for 17 weeks of observation after completion of the dosing schedule.

### Inclusion Criteria

Participants were selected for treatment based on the following criteria:
1. Male or female participants 18 to 70 years (inclusive) of age at Screening.
2. Body mass index ≥18.0 and ≤40.0 kg/m2.
3. Diagnosis of RA meeting the 2010 American College of Rheumatology/European League Against Rheumatism classification criteria for RA≥3 months before Screening (Appendix 2).
4. Use of methotrexate at 7.5 to 25 mg/week for ≥3 months, with stable dosing for ≥4 weeks, before randomization. Hydroxychloroquine/chloroquine and/or sulfasalazine are allowed if started ≥3 months before randomization and a stable dose is maintained after the Screening Visit.
5. A female participant is eligible if she is not pregnant (Section 9.2), not breastfeeding, and at least one of the following conditions applies:
   Not a woman of child-bearing potential (WOCBP) as defined in Section 9.2; OR
   A WOCBP who agrees to follow the contraceptive guidance in Section 9.2 through 6 months after the last study drug administration AND
   Agrees to refrain from donating eggs through 6 months after the last study drug administration.
6. A male participant must:
   Agree to (1) abstain from intercourse or (2) use contraception (as detailed in Section 9.2) during the Treatment Period and for at least 6 months after the last dose of study drug, or (3) be surgically sterile for the duration of the study; and
   Agree to refrain from donating sperm during the study period and for at least 6 months after the last dose of study drug.
7. A WOCBP must have a negative serum pregnancy test at Screening and a negative urine test before the first dose of study drug.
8. Capable of giving signed informed consent as described in Section 13.2 which includes compliance with the requirements and restrictions listed in the informed consent form (ICF) and in this protocol.
9. Willing to comply with all study protocol procedures and complete all study visits.

### Exclusion Criteria

Eligible participants did not meet any of the following criteria:
1. Inflammatory joint disease other than RA. Note: Current diagnosis of secondary Sjogren's Syndrome is permitted.
2. Surgery within 4 weeks before Screening or planned surgery during the clinical study.
3. History of any malignancy within 5 years, except for successfully treated nonmelanoma skin cancer or localized carcinoma in situ of the cervix.
4. Documented history of drug abuse in the previous 12 months before Screening (Days-28 to-1), or positive for urine drug screen for nonprescribed drugs other than cannabinoid at Screening.
5. Any condition considered by the investigator to make participation in the study inappropriate.
6. Donated blood (including component blood) or lost >400 mL within 1 month before Screening or received a transfusion within 3 months of Screening.
7. History of relevant allergies (including allergy to any murine or human-derived protein or immunoglobulin products, rubber or latex, or other allergies that in the opinion of the investigator make inclusion in the study inappropriate).
8. After the Screening Visit, corticosteroid use >10 mg/day (prednisone equivalent) or increase in dose.
9. Use before Screening of one or more of the following:
   a) Within 6 months: rituximab or similar major B-cell-depleting biologic therapy, or T-cell-depleting agent such as Campath® (alemtuzumab).
   b) Within 2 months: abatacept, leflunomide, infliximab, adalimumab, golimumab, certolizumab, etanercept, or any anti-TNF agent, tocilizumab, cyclosporine or other calcineurin inhibitor, azathioprine, mycophenolate mofetil, anakinra, immunoglobulin, Janus kinase (JAK) inhibitor, or blood products.
   c) Within 5 half-lives or 30 days, whichever is longer: agents (other than methotrexate, hydroxychloroquine/chloroquine, and sulfasalazine) considered by the investigator to have immunomodulating or immunosuppressive effects.
   d) Within 1 month: live or live-attenuated vaccines.
10. Currently enrolled in another interventional clinical study.
11. Positive for HIV infection, active hepatitis C, or hepatitis B (surface antigen positive or core antibody positive with surface antibody negative).
12. Quantiferon®-TB or T-SPOT® test positive for Mycobacterium tuberculosis (only one of these 2 tests needs to be performed).
13. Bacterial, viral, systemic fungal, parasitic, or opportunistic infection not resolved at least 14 days before Study Day 1 or expected to be treated with antibiotics during the Treatment Period, or history of recurrent infections.
14. Clinically significant blood chemistry abnormality at Screening, as indicated by ALT or AST≥2×ULN, TBIL≥1.5×ULN, or DBIL≥1.5×ULN.
15. Clinically significant hematologic abnormality at Screening, as indicated by WBC <2500/μL (2.5×109/L), hemoglobin <9 g/dL (90 g/L), or platelet count <75,000/μL (75×109/L).
16. Estimated glomerular filtration rate (eGFR)<30 mL/min/1.73 m2, using the Chronic Kidney Disease Epidemiology Collaboration (CKD-EPI) equation. eGFR according to the CKD-EPI equation=142×min (Scr/τ, 1)α×max (Scr/κ, 1)−1.200×0.9938 Age×1.012 [if female], where Scr is serum creatinine (in mg/dL), κ is 0.7 for females and 0.9 for males, α is −0.241 for females and −0.302 for males, min indicates the minimum of Scr/k or 1, max indicates the maximum of Scr/κ or 1, and age is in years (National Kidney Foundation 2021) (Levey, Stevens et al. 2009).
17. Clinically significant ECG abnormality, such as a QTcF interval >450 ms for male participants or >470 ms for female participants.
18. Employees or related personnel of the study site, the sponsor, or contract research organization.
19. Cohort has completed randomization of 8 participants while the participant is in the Screening Period.

### Objectives and Endpoints

The primary objective of the Part B study is to evaluate the safety and tolerability of multiple doses of anti-TNFα antibody 1 in participants with RA. The primary endpoint will be assessed based on the number of participants with TEAEs, serious TEAEs, and TEAEs leading to discontinuation of the dosing schedule.

Secondary objectives includes evaluating PK, immunogenicity, and pharmacodynamics (PD) in participants with RA following multiple doses of anti-TNFα antibody 1. Observed serum trough concentrations will be assessed prior to repeat-dose administration (or at the end of the dosing interval [tau] after the final dose) (C_{trough}) for all doses. For doses 1 and 3, the following will be assessed: Cₘₐₓ, Tₘₐₓ, AUC over the dosing interval and tau (AUCₜₐᵤ), accumulation ratio of Cₘₐₓ (ARCₘₐₓ), and accumulation ratio of AUC (ARAUC). For dose 3 only, AUC_{inf}, t12, AUCₗₐₛₜ, and CL/F, and V_{z}/F will be assessed. Additional secondary endpoints include serum anti-anti-TNFα antibody 1 antibody prevalence and incidence and cytokine/chemokine secretion in ex vivo stimulated whole blood (e.g., TNFα).

Exploratory objectives include evaluating PD effects of anti-TNFα antibody 1 on blood-biomarker levels. The exploratory endpoints include assessing at study visits the absolute level and change from baseline in Disease Activity Score 28 (DAS28) with C-reactive protein (CRP; DAS28-CRP), the proportion of participants with DAS28-CRP<2.6, the proportion of participants with DAS28-CRP<3.2, and the change from baseline in the following parameters: tender joint count (28 count and 68 count), swollen joint count (28 count and 66 count), high-sensitivity CRP (hs-CRP), patient's Global Assessment and physician's Global Assessment. Serum immune-mediated biomarkers (e.g., cytokines) and disease-associated biomarkers (e.g., rheumatoid factor [RF]) will also be assessed by study visit.

1. A stable formulation comprising an anti-TNFα antibody at a concentration of between 50 mg/mL and 200 mg/mL, wherein the anti-TNFα antibody comprises:
a light chain variable region comprising a LCDR1 of SEQ ID NO: 2, a LCDR2 of SEQ ID NO: 3, and a LCDR3 of SEQ ID NO: 4;
a heavy chain variable region comprising a HCDR1 of SEQ ID NO: 5, a HCDR2 of SEQ ID NO: 6, and a HCDR3 of SEQ ID NO: 7, and
an Fc region comprising M428L and N434S substitutions.

2. The stable formulation of claim 1, wherein the light chain variable region comprises SEQ ID NO: 11 and the heavy chain variable region comprises SEQ ID NO: 12.

3. The stable formulation of claim 1, wherein the anti-TNFα antibody comprises a light chain of SEQ ID NO:1 and a heavy chain of SEQ ID NO: 2.

4-5. (canceled)

6. The stable formulation of claim 1, wherein the concentration of the anti-TNFα antibody is 100 mg/mL.

7-12. (canceled)

13. The stable formulation of claim 1, wherein the formulation further comprises a histidine buffer at a concentration of 5 mM to 40 mM.

14-18. (canceled)

19. The stable formulation of claim 1, wherein the formulation comprises a pH of 5.0 to 6.0.

20-27. (canceled)

28. The stable formulation of claim 1, wherein the formulation further comprises a polyol at a concentration of 2% (w/v) to 10% (w/v).

29-30. (canceled)

31. The stable formulation of claim 28, wherein sorbitol is present at a concentration of 4.5% (w/v).

32. The stable formulation of claim 1, wherein the formulation further comprises an antioxidant.

33. (canceled)

34. The stable formulation of claim 32, wherein L-methionine is present at a concentration of 5 mM to 20 mM.

35. (canceled)

36. The stable formulation of claim 1, wherein the formulation further comprises a surfactant.

37-41. (canceled)

42. The stable formulation of claim 1, wherein the formulation is substantially free of salt.

43-45. (canceled)

46. The stable formulation of claim 1, comprising:
(i) 20 mM of histidine buffer;
(ii) 4.5% (w/v) of sorbitol;
(iii) 10 mM of L-methionine;
(iv) 0.02% (w/v) of polysorbate 80;
and
wherein the pH is 5.5.

47. The stable formulation of claim 46, wherein the formulation comprises an osmolality of between 310 mOsmol/kg and 410 mOsmol/kg.

48. (canceled)

49. The stable formulation of claim 1, wherein the concentration of the anti-TNFα antibody is 150 mg/mL.

50-51. (canceled)

52. The stable formulation of claim 1, wherein the anti-TNFα antibody is present as a monomer equal to or greater than 90%, equal to or greater than 91%, equal to or greater than 92%, equal to or greater than 93%, equal to or greater than 94%, equal to or greater than 95%, equal to or greater than 96%, equal to or greater than 97%, equal to or greater than 98%, equal to or greater than 99%, equal to or greater than 99.5%.

53. The stable formulation of claim 1, wherein the percentage of high molecular weight species is less than or equal to 5%, less than or equal to 4%, less than or equal to 3%, less than or equal to 2%, less than or equal to 1% or less than or equal to 0.5%.

54-60. (canceled)

61. A method of treating an autoimmune disease in a subject comprising, administering to the subject an anti-TNFα antibody at a dose of at least 20 mg no more frequently than once every other week,
wherein the anti-TNFα antibody comprises
a light chain variable region comprising a LCDR1 of SEQ ID NO: 2, a LCDR2 of SEQ ID NO: 3, and a LCDR3 of SEQ ID NO: 4;
a heavy chain variable region comprising a HCDR1 of SEQ ID NO: 5, a HCDR2 of SEQ ID NO: 6, and a HCDR3 of SEQ ID NO: 7, and
an Fc region comprising M428L and N434S substitutions.

62-68. (canceled)

69. A method of storing a stable formulation, comprising storing a formulation comprising an anti-TNFα antibody at 2-8° C. for at least 3 months, wherein the anti-TNFα antibody is at a concentration of between 50 mg/mL and 200 mg/mL and wherein the anti-TNFα antibody comprises:
a light chain variable region comprising a LCDR1 of SEQ ID NO: 2, a LCDR2 of SEQ ID NO: 3, and a LCDR3 of SEQ ID NO: 4;
a heavy chain variable region comprising a HCDR1 of SEQ ID NO: 5, a HCDR2 of SEQ ID NO: 6, and a HCDR3 of SEQ ID NO: 7, and
an Fc region comprising M428L and N434S substitutions.

70. The method of claim 69, wherein storing the formulation results in less than 5% aggregates, and greater than 90% intact antibody in the formulation.
